(19) United States
(12) Patent Application Publication
(10) Pub. No.: US 2026/0196302 A1
(43) Pub. Date: Jul. 9, 2026

(54) METHODS AND SYSTEMS FOR DETECTING RECOMBINATION EVENTS

(71) Applicant: Illumina, Inc., San Diego, CA (US)

(72) Inventors: Jonathan Robert Belyeu, San Diego, CA (US); Xiao Chen, Richland, WA (US); Eric Edward Roller, San Diego, CA (US)

(21) Appl. No.: 18/868,009

(22) PCT Filed: Jul. 5, 2023
(86) PCT No.: PCT/US2023/026931

§ 371 Date: Nov. 21, 2024

Related U.S. Application Data
(60) Provisional application No. 63/367,896, filed on Jul. 7, 2022.

(51) Int. Cl. G16B 30/10; G16B 20/10; G16B 20/20

(52) U.S. Cl. CPC G16B 30/10; G16B 20/10; G16B 20/20

(57) ABSTRACT

Disclosed herein are systems, devices, and methods for identifying recombinant variants (such as duplication, deletion, and/or gene conversion variants) of genes such as the CYP21A2 gene or the CYP21A1P gene, the copy numbers of the RCCX region, and candidate haplotypes. Also disclosed herein are systems, devices, and methods for detecting one or more single-nucleotide variants or indels in a RCCX region in a nucleic acid sample.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims priority to U.S. Provisional Application No. 63/367,896, filed Jul. 7, 2022, which is hereby incorporated by reference in its entirety.

### BACKGROUND

### Field

The disclosed technology relates to the field of nucleic acid sequencing. More particularly, the disclosed technology relates to detecting a recombination event between a CYP2142 gene and a CYP21A1P gene in a nucleic acid sample.

### Description of the Related Art

CYP2142 encodes 21-hydroxlyase, a cytochrome P450 enzyme that aids in adrenal regulation of the cortisol and aldosterone hormones. These hormones play a number of roles, including in regulating salt retention in the kidneys. Inactivation of CYP21A2 is responsible for 95% of 21-hydroxlyase CAH cases, which can take one of three forms. The first form is salt-wasting CAH, which is the most severe and in which complete deficiency of CYP21A2 leads to very low levels of aldosterone synthesis and thus decreased sodium retention. Symptoms can be very severe, including dehydration, diarrhea, vomiting, and adrenal crisis, and can lead to death. Low cortisol levels also play a developmental role and can lead to virilization. The second form is simple virilizing CAH, which is a more moderate form, and is caused by decreased CYP21A2 activity without complete gene deficiency. This form generally avoids the most severe and life-threatening symptoms, but still typically presents virilization and developmental challenges. The third form is non-classic CAH, with similar symptoms to simple virilizing CAH. Non-classic CAH is characterized by higher aldosterone and cortisol hormone levels, resulting in milder symptom severity. Due to the lesser phenotypic impact, non-classic CAH is more difficult to diagnose.

CYP21A2 lies within a 30 kilobase segmental duplication, in the major histocompatibility complex (MHC) class III region. The repeat is commonly referred to as RCCX and contains part or all of four genes: STK19, C4A/C4B, CYP21A2, and TNXB. The RCCX repeat canonically exists as two modules with nearly identical sequences. The first module contains the end of the STK19 gene, an active C44 gene, and two inactive pseudogenes: CYP21A1P and TNXA. The second module contains C4B, CYP21A2, and the end of TNXB, all active genes with important roles in human health.

The high sequence homology of the RCCX region drives a high rate of non-allelic homologous recombination. These recombination events may occur at any point within the repeat. If the breakpoints of a recombination event lie within the regions of CYP21A2, a chimeric gene fusion is created with part of the sequence of the pseudogene and part of the sequence of the gene. Despite sequence similarity between gene and pseudogene of about 98%, these chimeric fusion genes may be partially or entirely inactivated by the introduction of a few small variants from the pseudogene into the gene. These may be considered partial gene conversions. CYP21A2 is also subject to more canonical gene conversion variants of partial gene sequences, perhaps due to template switching during break repair in synthesis.

If the recombination breakpoints for a deletion occur outside the gene, it may be entirely deleted from the resulting chimeric RCCX module, leaving only CYP21A1P. This heterozygous CYP21A2 deletion creates a carrier status and will result in phenotypic impacts if later co-inherited with another deficient allele.

### SUMMARY

In one aspect, disclosed herein are computer-implemented methods of detecting a recombination event between a CYP21A2 gene and a CYP21A1P gene in a nucleic acid sample. In some embodiments, the methods include receiving sequence reads which align to a RCCX region of a human genome in the nucleic acid sample; estimating a copy number of the RCCX region of the human genome in the nucleic acid sample from the aligned sequence reads; constructing one or more candidate haplotypes by phasing a plurality of sequence reads which align to a CYP21A2 gene or a CYP21A1P gene of the human genome and which include at least two pre-determined differentiating sites of the CYP21A2 gene and the CYP21A1P gene; and detecting a recombination event between the CYP21A2 gene and the CYP21A1P gene based on the estimated copy number of the RCCX region of the human genome and based on the one or more candidate haplotypes.

In some embodiments, the one or more candidate haplotypes cover one or more breakpoints of the recombination event. In some embodiments, constructing the one or more candidate haplotypes includes identifying at least one seed sequence read from the plurality of sequence reads. In some embodiments, the seed sequence read is selected from a 5′ seed sequence read, a center sequence read, and a 3′ seed sequence read. In some embodiments, constructing the one or more candidate haplotypes includes iteratively extending at least one seed sequence read in either a 5′ direction or a 3′ direction by aligning the sequence reads using the pre-determined differentiating sites.

In some embodiments, estimating a copy number of the RCCX region of the human genome includes counting sequence reads which align to the RCCX region of the human genome. In some embodiments, estimating a copy number of the RCCX region of the human genome includes counting sequence reads which align to a C4A gene, a CYP21A1P gene, a TNXA gene, a C4B gene, a CYP21A2 gene, or a TNXB gene in the human genome. In some embodiments, estimating a copy number of the RCCX region of the human genome includes counting sequence reads which align to a region corresponding to positions chr6:32024461-chr6:32043719 of reference genome hg38, chr6:31991723-chr6:32010985 of reference genome hg38, chr6:31992238-chr6:32011496 of reference genome hg19, or chr6:31959500-chr6:31978762 of reference genome hg19.

In some embodiments, estimating the copy number includes normalizing the count of the sequence reads which align to the ROCK region of the human genome. In some embodiments, estimating the copy number includes binning the normalized count of the sequence reads which align to the RCCX region of the human genome using a Gaussian mixture model.

In some embodiments, the methods and systems disclosed further include a step of making a variant call at a pre-determined differentiating site of the plurality of pre-determined differentiating sites. In some embodiments, the methods and systems disclosed further include a step of making a variant call for the recombination event. In some embodiments, the methods and systems disclosed further include a step of creating a digital file including a variant call. In some embodiments, the methods and systems disclosed further include a step of creating a digital file including one or more candidate haplotypes.

In some embodiments, the plurality of pre-determined differentiating sites include a site corresponding to a position selected from chr6:32038514, chr6:32038844, chr6:32039015, chr6:32039081, chr6:32039128, chr6:32039132, chr6:32039143, chr6:32039426, chr6:32039548, chr6:32039802, chr6:32039807, chr6:32039810, chr6:32039816, chr6:32040110, chr6:32040182, chr6:32040216, chr6:32040421, or chr6:32040535 of the CYP21A2 gene or a corresponding position in pseudogene CYP21A1P, in reference genome hg38. In some embodiments, the plurality of pre-determined differentiating sites include a site corresponding to a position selected chr6:32006291, chr6:32006621, chr6:32006792, chr6:32006858, chr6:32006905, chr6:32006909, chr6:32006920, chr6:32007203, chr6:32007325, chr6:32007579, chr6:32007584, chr6:32007587, chr6:32007593, chr6:32007887, chr6:32007959, chr6:32007993, chr6:32008198, or chr6:32008312 of the CYP21A2 gene or a corresponding position in pseudogene CYP21A1P, in reference genome hg19.

In another aspect, disclosed herein are computer-implemented methods of detecting one or more single-nucleotide variants or indels in a RCCX region in a nucleic acid sample. In some embodiments, the methods include: determining sequence reads from the nucleic acid sample; obtaining sequence reads which align to a site of a single-nucleotide variant or indel in a CYP21A2 gene or a CYP21A1P gene of a human genome in the nucleic acid sample; counting sequence reads which contain a base corresponding to an alternative allele at the site of the single-nucleotide variant or indel, wherein counting sequence reads comprises counting sequence reads which align to the CYP21A2 gene and sequence reads which align to the CYP21A1P gene; and creating a digital file including a variant call corresponding to the single-nucleotide variant or indel, wherein the variant call is not specific to the CYP21A2 gene or the CYP21A1P gene.

In some embodiments, the one or more single-nucleotide variants or indels include NM_000500.9:c.60G>A, NM_000500.9:c.92C>A, NM_000500.9:c.111del, NM_000500.9:c.159 160del, NM_000500.9:c.169G>A, NM_000500.9:c.274A>G, NM_000500.9:c.332_339del, NM_000500.9:c.418G>A, NM_000500.9:c.421G>A, NM_000500.9:c.515T>A, NM_000500.9:c.710 719delinsACGAGGAGAA, NM_000500.9:c.850A>G, NM_000500.9:c.922T>G, NM_000500.9:c.874G>>A, NM_000500.9:c.952C>T=, NM_000500.9:c.955C>G, NM_000500.9:c.923 924dup, NM_000500.9:c.1051G>A, NM_000500.9:c.1066C>T=, NM_000500.9:c.1042G>A, NM_000500.9:c.1096C>T, NM_000500.9:c.1118G>A, NM_000500.9:c.1070G>A, NM_000500.9:c.1226G>A, NM_000500.9:c.1273G>A, NM_000500.9:c.1136T>A, NM_000500.9:c.1279C>T, NM_000500.9:c.1357C>T=, NM_000500.9:c.1274G>T, NM_000500.9:c.1360C>T, NM_000500.9:c.1444C>T, NM_000500.9:c.1450dup, or NM_000500.9:c.1451G>A.

In another aspect, disclosed herein are electronic systems for detecting a recombination event between a CYP21A2 gene and a CYP21A1P gene in a nucleic acid sample. In some embodiments, the systems include a processor configured to perform a method comprising: receiving sequence reads which align to a RCCX region of a human genome in the nucleic acid sample; estimating a copy number of the RCCX region of the human genome in the nucleic acid sample from the aligned sequence reads; constructing one or more candidate haplotypes by phasing a plurality of sequence reads which align to a CYP21A2 gene or a CYP21A1P gene of the human genome and which include at least two pre-determined differentiating sites of the CYP21A2 gene and the CYP21A1P gene; and detecting a recombination event between the CYP21A2 gene and the CYP21A1P gene based on the estimated copy number of the RCCX region of the human genome and based on the one or more candidate haplotypes.

In some embodiments, the processor is configured to perform a method comprising detecting a recombination event between the CYP21A2 gene and the CYP21A1P gene based on the estimated copy number of the RCCX region of the human genome and based on the one or more candidate haplotypes.

In some embodiments, the one or more candidate haplotypes cover one or more breakpoints of the recombination event. In some embodiments, constructing the one or more candidate haplotypes includes identifying at least one seed sequence read from the plurality of sequence reads. In some embodiments, the seed sequence read is selected from a 5′ seed sequence read, a center sequence read, and a 3′ seed sequence read. In some embodiments, constructing the one or more candidate haplotypes includes iteratively extending at least one seed sequence read in either a 5′ direction or a 3′ direction by aligning the sequence reads using the pre-determined differentiating sites.

In some embodiments, estimating a copy number of the RCCX region of the human genome comprises counting sequence reads which align to the RCCX region of the human genome.

In a further aspect, disclosed herein are electronic systems for detecting one or more single-nucleotide variants or indels in a RCCX region in a nucleic acid sample. In some embodiments, the systems include a processor configured to perform a method including: determining sequence reads from the nucleic acid sample; obtaining sequence reads which align to a site of a single-nucleotide variant or indel in a CYP21A2 gene or a CYP21A1P gene of a human genome in the nucleic acid sample; counting sequence reads which contain a base corresponding to an alternative allele at the site of the single-nucleotide variant or indel, wherein counting sequence reads comprises counting sequence reads which align to the CYP21A2 gene and sequence reads which align to the CYP21A1P gene; and creating a digital file including a variant call corresponding to the single-nucleotide variant or indel, wherein the variant call is not specific to the CYP21A2 gene or the CYP21A1P gene.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A schematically illustrates the RCCX region and RCCX modules.

FIG. 1B schematically illustrates recombination events within the RCCX region.

FIG. 2A is a block diagram that schematically illustrates methods of detecting a recombination event between a CYP21A2 gene and a CYP21A1P gene in a nucleic acid sample.

FIG. 2B is a block diagram that further schematically illustrates a process of constructing one or more candidate haplotypes.

FIG. 3 schematically illustrates an embodiment of a candidate haplotype construction process.

FIG. 4A is a block diagram of an exemplary sequencing system that may be used to perform the disclosed methods.

FIG. 4B is a block diagram of an exemplary computing device that may be used in connection with the exemplary sequencing system of FIG. 4A.

FIG. 5 schematically illustrates recombinant haplotypes constructed in a congenital adrenal hyperplasia (CAH) case trio.

FIG. 6 graphically illustrates a comparison of RCCX module copy number estimation with copy number calls from Bionano optical mapping.

### DETAILED DESCRIPTION

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

### Overview

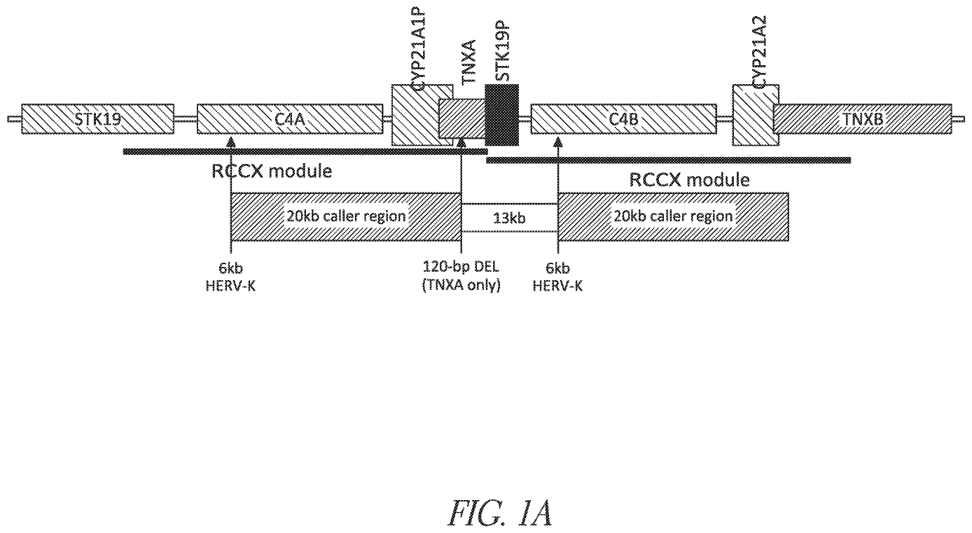

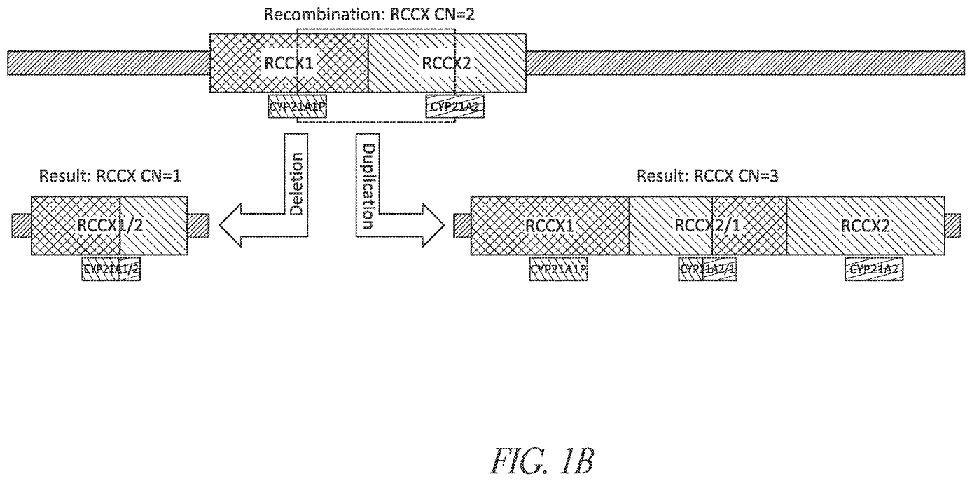

CYP21A2 lies within the RCCX region, which is schematically illustrated in FIG. 1A. Recombination events occur at a high rate within the RCCX region due to high sequence homology. For example, a deletion event and a duplication event occurring between CYP21A2 and CYP21A1P are schematically depicted in FIG. 1B. Recombination events in the RCCX region, such as between CYP21A2 and CYP21A1P, may be difficult to detect due to the high sequence homology between the CYP21A2 and CYP21A1P genes. For example, gene conversion variants may be difficult to detect, as sequence reads over a gene conversion boundary may contain the allele from the alternate RCCX module at the gene conversion site and may be preferentially mapped to the wrong gene.

Other small variants (single-nucleotide and insertion/deletion events) can also lead to decreased CYP21A2 activity. These variants may occur in regions of the CYP21A2 gene where the nucleotide sequence is identical to the CYP21A1P pseudogene, which may make variant detection extremely challenging. This is because reads sequenced from either the gene or pseudogene may lack identifying markers, meaning that during an assembly process following sequencing they may be randomly assigned to the wrong gene. This can result in weak and ambiguous evidence for a variant in either location, which can mean missed or low-confidence variant calls.

Due to this combination of factors, it has been difficult to accurately determine sequence information for the CYP21A2 gene or CYP21A1P gene using whole genome sequence (WGS) data. The methods and systems of the present disclosure overcome challenges of sequence homology between the CYP21A2 gene and the CYP21A1P pseudogene to detect multiple types of gene variants in this genomic region. Such gene variants may include: small variants, gene conversions, and recombination-derived whole-gene deletions or duplications.

Described herein are methods and systems for detecting a recombination event between a CYP21A2 gene and a CYP21A1P gene in a nucleic acid sample taken from one or more subjects. The disclosed systems and methods for detecting a recombination event between a CYP21A2 gene and a CYP21A1P in a nucleic acid sample were found to improve the specificity and sensitivity of detecting recombination event(s) between a CYP21A2 gene and a CYP21A1P and of variant calling in the RCCX region in the nucleic acid sample.

In some embodiments, the disclosed systems and methods include receiving sequence reads which align to a RCCX region found in a biological sample taken from a subject. Once the sequence reads are received, a copy number of the RCCX region can be estimated. Estimating the RCCX copy number may include counting the sequence reads that align to the RCCX region of a reference genome.

The disclosed systems and methods may then construct one or more candidate haplotypes by phasing a plurality of sequence reads which align to a CYP21A2 gene or a CYP21A1P gene of the human genome and which include at least two pre-determined differentiating sites of the CYP21A2 gene and the CYP21A1P gene. These pre-determined differentiating sites may include positions in the nucleic acid sequence of the CYP21A2 gene or a corresponding position in the CYP21A1P gene which include at least one base that differs between the CYP21A2 gene and the CYP21A1P gene, and which difference is pre-determined to be fixed in a population. Thus, these pre-determined differentiating sites may be used to determine whether a particular sequence read corresponds to the CYP21A2 gene or the CYP21A1P gene, including detecting a recombination event between a CYP21A2 gene and a CYP21A1P gene.

In some embodiments, the disclosed systems and methods detect a recombination event between the CYP21A2 gene and the CYP21A1P gene based on the estimated copy number of the RCCX region of the human genome and based on the one or more candidate haplotypes. For example, the disclosed methods and systems may detect a recombination event, such as a gene conversion, duplication, or deletion based on an estimated RCCX copy number and/or based on detection of a transition from CYP21A2-specific bases to CYP21A1P-specific bases (or vice-versa) along the pre-determined differentiating sites in one or more candidate haplotypes.

The disclosed systems and methods can improve the recall (also known as sensitivity, the percentage of true variants that are correctly detected) of single nucleotide polymorphisms (SNPs) generated by a recombination event between a CYP21A2 gene and a CYP21A1P gene by 20%, 50%, 80%, 100% or more.

### Definitions

Unless defined otherwise, technical and scientific terms used herein have the same meaning as commonly understood by one of ordinary skill in the art to which the present disclosure belongs. See, for example, Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides may be natural nucleotides, but it is to be further understood that non-natural nucleotides, modified nucleotides or analogs of the aforementioned nucleotides can also be used.

As used herein, “base” or “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-cthanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrotbymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 485-494, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

A “genome” refers to the complete genetic information of an organism or virus, expressed in nucleic acid sequences.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at nebi.nlm.nih.gov. In various embodiments, the reference sequence is significantly larger than the reads that are aligned to it. For example, it may be at least about 100 times larger, or at least about 1000 times larger, or at least about 10,000 times larger, or at least about 105 times larger, or at least about 106 times larger, or at least about 107 times larger. In one example, the reference sequence is that of a full-length genome. Such sequences may be referred to as genomic reference sequences. For example, the reference sequence can be a reference human genome sequence, such as hg19 (for example, available at GenBank assembly accession GCA_000001405.1) or hg38 (for example, available at GenBank assembly accession GCA_000001405.15). In another example, the reference sequence is limited to a specific human chromosome such as chromosome 13. In some embodiments, a reference Y chromosome is the Y chromosome sequence from human genome version hg19. Such sequences may be referred to as chromosome reference sequences. Other examples of reference sequences include genomes of other species, as well as chromosomes, sub-chromosomal regions (such as strands), etc., of any species. In various embodiments, the reference sequence is a consensus sequence or other combination derived from multiple individuals. However, in certain applications, the reference sequence may be taken from a particular individual.

The term “nucleic acid sample” herein refers to a sample, typically derived from a biological fluid, cell, tissue, organ, or organism, comprising a nucleic acid or a mixture of nucleic acids comprising at least one nucleic acid sequence that is to be screened for copy number variation. In certain embodiments the nucleic acid sample comprises at least one nucleic acid sequence whose copy number is suspected of having undergone variation. Such samples may include, but are not limited to sputum/oral fluid, amniotic fluid, blood, a blood fraction, or fine needle biopsy samples (such as surgical biopsy, fine needle biopsy, etc.), urine, peritoneal fluid, pleural fluid, and the like. Although the sample is often taken from a human subject (such as a patient), the sample may be from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (such as namely, a sample that is not subjected to any such pretreatment method(s)). Such “treated” or “processed” samples are still considered to be biological “test” samples with respect to the methods described herein.

The term “read” or “sequence read” (or sequencing reads) refer to a sequence obtained from a portion of a nucleic acid sample. A read may be represented by a string of nucleotides sequenced from any part or all of a nucleic acid molecule. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (such as at least about 25 bp) that can be used to identify a larger sequence or region, for example, that can be aligned and specifically assigned to a chromosome or genomic region or gene. For example, a sequence read may be a short string of nucleotides (such as 20-150 bases) sequenced from a nucleic acid fragment, a short string of nucleotides at one or both ends of a nucleic acid fragment, or the sequencing of the entire nucleic acid fragment that exists in the biological sample. Sequence reads may be obtained by any method known in the art. For example, a sequence read may be obtained in a variety of ways, such as using sequencing techniques or using probes, such as in hybridization arrays or capture probes, or amplification techniques, such as the polymerase chain reaction (PCR) or linear amplification using a single primer or isothermal amplification. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

The term “sequencing depth,” as used herein, generally refers to the number of times a locus is covered by a sequence read aligned to the locus. The locus may be as small as a nucleotide, or as large as a chromosome arm, or as large as the entire genome. Sequencing depth can be expressed as 50×, 100×, etc., where “×” refers to the number of times a locus is covered with a sequence read. Sequencing depth can also be applied to multiple loci, or the whole genome, in which case ×can refer to the mean number of times the loci or the haploid genome, or the whole genome, respectively, is sequenced. When a mean depth is quoted, the actual depth for different loci included in the dataset spans over a range of values. Ultra-deep sequencing can refer to at least 100× in sequencing depth.

As used herein, the terms “aligned,” “alignment,” or “aligning” refer to the process of comparing a read or tag to a reference sequence and thereby determining the likelihood of the reference sequence contains the read sequence. If the reference sequence contains the read, the read may be mapped to the reference sequence or, in certain embodiments, to a particular location in the reference sequence. For example, the alignment of a read to the reference sequence for human chromosome 13 will tell the likelihood of the read is present in the reference sequence for chromosome 13. In some cases, an alignment additionally indicates a location where the read or tag maps to in the reference sequence. For example, if the reference sequence is the whole human genome sequence, an alignment may indicate that a read is present on chromosome 13, and may further indicate that the read is on a particular strand and/or site of chromosome 13. A “site” may be a unique position on a polynucleotide sequence or a reference genome (i.e. chromosome ID, chromosome position and orientation). In some embodiments, a site may provide a position for a residue, a sequence tag, or a segment on a sequence.

Aligned reads or tags are one or more sequences that are identified as a match in terms of the order of their nucleic acid molecules to a known sequence from a reference genome. Alignment can be done manually, although it is typically implemented by a computer algorithm, as it would be impossible to align reads in a reasonable time period for implementing the methods disclosed herein. The matching of a sequence read in aligning can be a 100% sequence match or less than 100% (non-perfect match).

Alignment may be performed by modifications and/or combinations of methods such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemchl, SeqMap, Shrec, SHRIMP, SLIDER, SOAP, SOAP2, SOAP3 and SOAP3-dp, SOCS, SSAHA and SSAHA2, Stampy, STORM, Subread and Subjunc, Taipan, UGENE, VelociMapper, XpressAlign, and ZOOM.

The term “mapping” used herein refers to specifically assigning a sequence read to a larger sequence, such as a reference genome, by alignment.

A “genetic variation” or “genetic alteration” refers to a particular genotype present in certain individuals, and often a genetic variation is present in a statistically significant sub-population of individuals. The presence or absence of a genetic variance can be determined using a method or apparatus described herein. In certain embodiments, the presence or absence of one or more genetic variations is determined according to an outcome provided by methods and apparatuses described herein. In some embodiments, a genetic variation is a chromosome abnormality (such as aneuploidy), partial chromosome abnormality or mosaicism, each of which is described in greater detail herein. Non-limiting examples of genetic variations include one or more deletions (such as micro-deletions), duplications (such as micro-duplications), insertions, mutations, polymorphisms (such as single-nucleotide polymorphisms), fusions, repeats (such as short tandem repeats), distinct methylation sites, distinct methylation patterns, the like and combinations thereof. An insertion, repeat, deletion, duplication, mutation or polymorphism can be of any length, and in some embodiments, is about 1 base or base pair (bp) to about 250 megabases (Mb) in length. In some embodiments, an insertion, repeat, deletion, duplication, mutation or polymorphism is about 1 base or base pair (bp) to about 1,000 kilobases (kb) in length (for example about 10 bp, 50 bp, 100 bp, 500 bp, 1 kb, 5 kb, 10 kb, 50 kb, 100 kb, 500 kb, or 1000 kb in length).

A genetic variation is sometimes a deletion. In certain embodiments a deletion is a mutation (such as a genetic aberration) in which a part of a chromosome or a sequence of DNA is missing. A deletion is often the loss of genetic material. Any number of nucleotides can be deleted. A deletion can comprise the deletion of one or more entire chromosomes, a segment of a chromosome, an allele, a gene, an intron, an exon, any non-coding region, any coding region, a segment thereof or combination thereof. A deletion can comprise a microdeletion. A deletion can comprise the deletion of a single base.

A genetic variation is sometimes a genetic duplication. In certain embodiments a duplication is a mutation (such as a genetic aberration) in which a part of a chromosome or a sequence of DNA is copied and inserted back into the genome. In certain embodiments a genetic duplication (i.e. duplication) is any duplication of a region of DNA, In some embodiments a duplication is a nucleic acid sequence that is repeated, often in tandem, within a genome or chromosome. In some embodiments a duplication can comprise a copy of one or more entire chromosomes, a segment of a chromosome, an allele, a gene, an intron, an exon, any non-coding region, any coding region, segment thereof or combination thereof. A duplication can comprise a microduplication. A duplication sometimes comprises one or more copies of a duplicated nucleic acid. A duplication sometimes is characterized as a genetic region repeated one or more times (such as repeated 1, 2, 3, 4, 5, 6, 7, 8, 9 or 10 times). Duplications can range from small regions (thousands of base pairs) to whole chromosomes in some instances. Duplications frequently occur as the result of an error in homologous recombination or due to a retrotransposon event. Duplications have been associated with certain types of proliferative diseases. Duplications can be characterized using genomic microarrays or comparative genetic hybridization (CGH).

A genetic variation is sometimes an insertion. An insertion is sometimes the addition of one or more nucleotide base pairs into a nucleic acid sequence. An insertion is sometimes a microinsertion. In certain embodiments an insertion comprises the addition of a segment of a chromosome into a genome, chromosome, or segment thereof. In certain embodiments an insertion comprises the addition of an allele, a gene, an intron, an exon, any non-coding region, any coding region, segment thereof or combination thereof into a genome or segment thereof. In certain embodiments an insertion comprises the addition (i.e., insertion) of nucleic acid of unknown origin into a genome, chromosome, or segment thereof. In certain embodiments an insertion comprises the addition (i.e. insertion) of a single base.

A genetic variation sometimes includes copy number variations, i.e., variations in the number of copies of a nucleic acid sequence present in a test sample in comparison with the copy number of the nucleic acid sequence present in a reference sample. In certain embodiments, the nucleic acid sequence is 1 kb or larger. In some cases, the nucleic acid sequence is a whole chromosome or significant portion thereof. A copy number variant may refer to the sequence of nucleic acid in which copy-number differences are found by comparison of a nucleic acid sequence of interest in test sample with an expected level of the nucleic acid sequence of interest. For example, the level of the nucleic acid sequence of interest in the test sample is compared to that present in a qualified sample. Copy number variants/variations may include deletions, including microdeletions, insertions, including microinsertions, duplications, multiplications, and translocations. CNVs encompass chromosomal aneuploidies and partial aneuploidies.

“Phasing” refers to analyzing linkage information between sequence reads of a nucleic acid to determine whether two subsequences of a nucleic acid (such as alleles or variants) are located on a single chromosome or two separate chromosomes (for example, a maternally or paternally inherited chromosome).

### Embodiments of Methods and Systems of Detecting a Recombination Event Between a CYP21A2 Gene and a CYP21A1P Gene

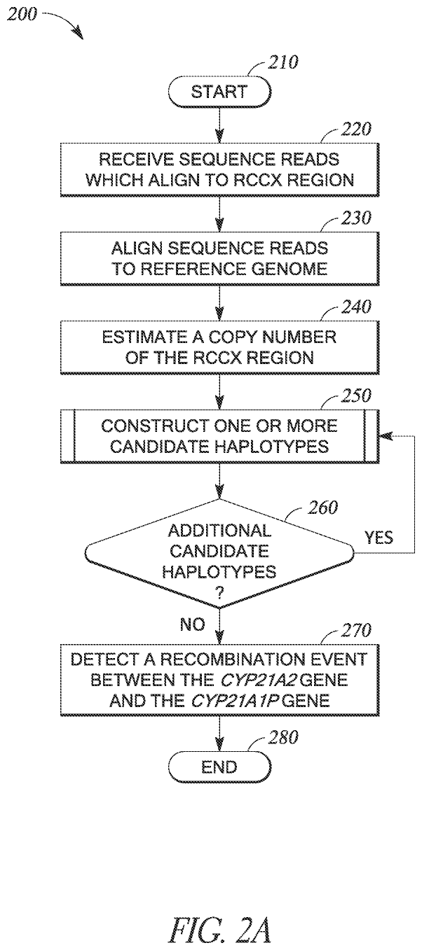

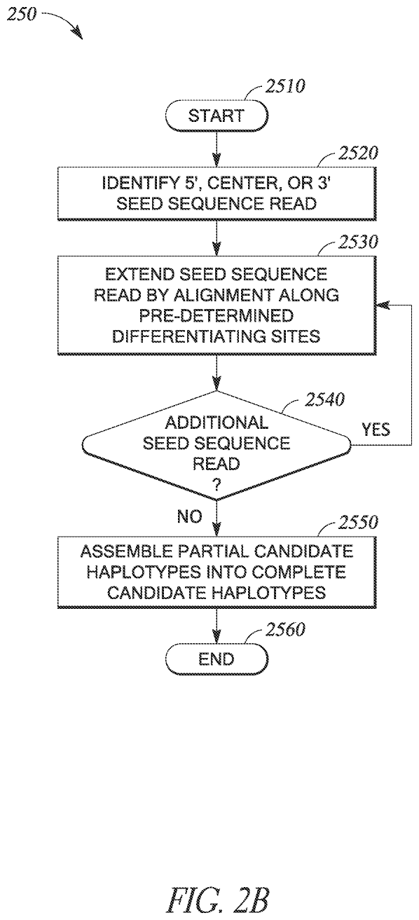

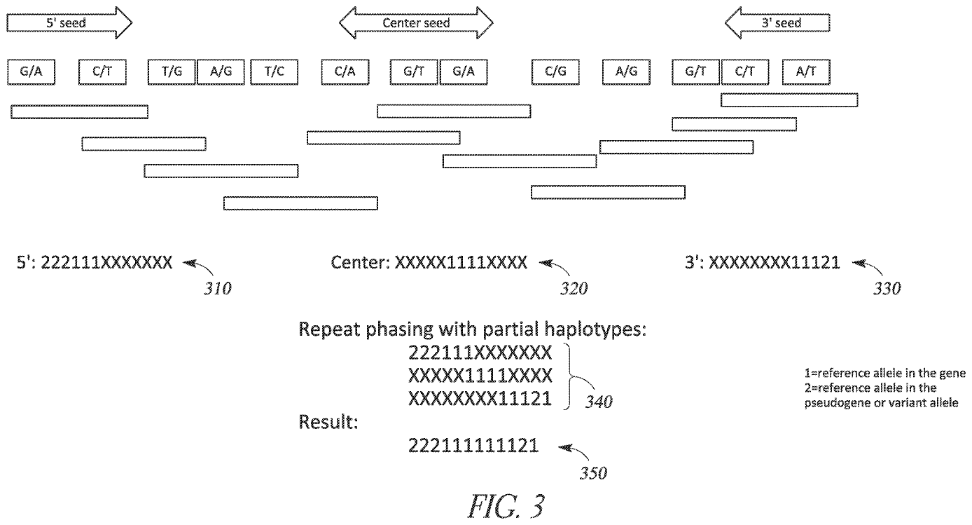

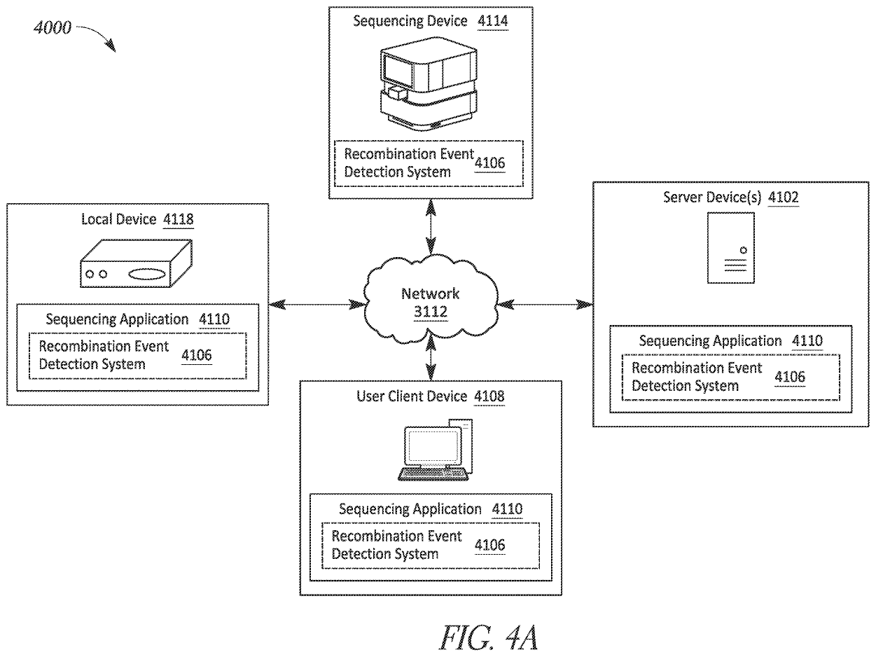

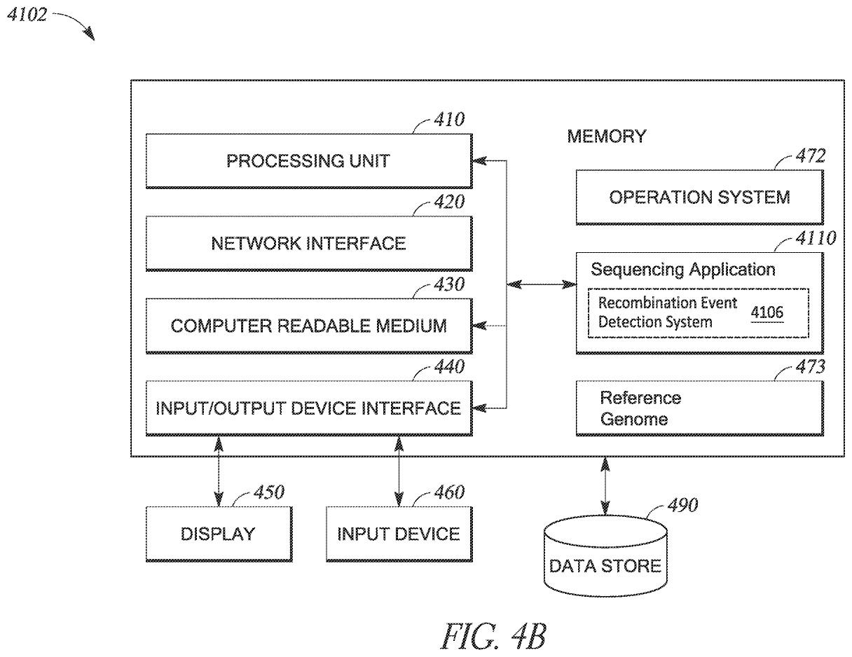

FIG. 2 is a block diagram that schematically illustrates an exemplary method 200 of detecting a recombination event between a CYP21A2 gene and a CYP21A1P gene in a nucleic acid sample. In some embodiments, the method 200 is implemented on a computer. The method 200 may be embodied in a set of executable program instructions stored on a computer-readable medium, such as one or more disk drives, of a computing system. For example, the server device 4102 shown in FIGS. 4A and 3B and described in greater detail below can execute a set of executable program instructions to implement the method 200. When the method 200 is initiated, the executable program instructions can be loaded into a memory, such as RAM, and executed by one or more processors of a server device 4102. Although the method 200 is described with respect to the server device 4102 shown in FIG. 4B, the description is illustrative only and is not intended to be limiting. In some embodiments, the method 200 or portions thereof may be performed serially or in parallel by multiple computing systems.

As shown in FIG. 2A, the method 200 for detecting a recombination event between a CYP21A2 gene and a CYP21A1P gene in a nucleic acid sample may start from start block 210. The method 200 may proceed to block 220, wherein sequence reads which align to a RCCX region of a human genome in the nucleic acid sample are received. The method may next proceed to block 230, wherein sequence reads are aligned to a reference genome, such as over the RCCX region. Next, the method 200 may proceed to block 240, wherein a copy number of the RCCX region of the human genome in the nucleic acid sample is estimated from the aligned sequence reads. Next, the method 200 may proceed to process block 250, wherein one or more candidate haplotypes are constructed. The methods performed within the process block 250 are described in further detail with respect to FIG. 2B. Next, the method 200 may proceed to decision state 260, wherein the system may decide whether there are additional candidate haplotypes to be constructed. If there are additional candidate haplotypes to be constructed, the method 200 may return to block 250 and the method may proceed as previously described. If there are no additional candidate haplotypes to be constructed, the method 200 may proceed to block 270, wherein a recombination event between the CYP21A2 gene and the CYP21A1P gene is detected. The method 200 may end at end block 280.

FIG. 2B is a block diagram that further illustrates the methods taking place within the process block 250 described above, wherein one or more candidate haplotypes are constructed. As shown in FIG. 2B, the method of process block 250 may start from start block 2510. The method of process block 250 may proceed to block 2520, wherein a 5′ seed sequence read, a center sequence read, or a 3′ seed sequence read is identified. The method of process block 250 may proceed to block 2530, wherein the seed sequence read is extended by alignment along pre-determined differentiating sites. The method of process block 250 may proceed to decision state 2540, wherein the system may decide whether there are additional seed sequence reads to be extended. If there are additional seed sequence reads to be extended, the workflow may return to block 2520 and the workflow may proceed as previously described. If there are no additional seed sequence reads to be extended, the workflow may proceed to block 2550, wherein partial candidate haplotypes are assembled into complete candidate haplotypes. The method of process block 250 may end at end block 2560.

Receiving Sequence Reads which Align to a RCCX Region

In some embodiments, the methods and systems disclosed herein include a step of receiving a plurality of sequence reads which align to a RCCX region of a human genome in the nucleic acid sample, for example as depicted in block 220 of FIG. 2A. In some embodiments, the sequence reads are generated from a sample obtained from a subject.

In some embodiments, the RCCX region includes two RCCX modules. For example, the RCCX modules have nearly identical sequences. In some embodiments, each RCCX module is about 10 kb, about 15 kb, about 20 kb, about 25 kb, about 30 kb (or a range constructed from any of these values) in length. In some embodiments, each RCCX module is about 20 kb in length. In some embodiments, each RCCX module is separated by about 5 kb, about 6 kb, about 7 kb, about 8 kb, about 9 kb, about 10 kb, about 11 kb, about 12 kb, about 13 kb, about 14 kb, about 15 kb, about 16 kb, about 17 kb, about 18 kb, about 19 kb, about 20 kb, about 25 kb, about 30 kb (or a range constructed from any of these values). In some embodiments, each RCCX module is separated by about 13 kb.

In some embodiments, the first RCCX module includes an end of a STK19 gene, a C4A gene, a CYP21A1P gene, and a TNXA gene. In some embodiments, the second RCCX module includes a C4B gene, a CYP21A2 gene, and an end of a TNXB gene. In some embodiments, the first RCCX module includes a HERV-K retrotransposon insertion in a C4A gene. In some embodiments, the HERV-K retrotransposon insertion is about 6.4 kb in length. In some embodiments, the second RCCX module includes a HERV-K retrotransposon insertion in a C4B gene. In some embodiments, the HERV-K retrotransposon insertion is about 6.4 kb in length. In some embodiments, the first RCCX module covers the site of a 120 bp deletion in TNXA gene as compared to TNXB gene.

In some embodiments, the RCCX region includes a region corresponding to positions chr6:32024461-chr6:32043719 of reference genome hg38, chr6:31991723-chr6:32010985 of reference genome hg38, chr6:31992238-chr6:32011496 of reference genome hg19, and/or chr6:31959500-chr6:31978762 of reference genome hg19. For example, in some embodiments, the RCCX region includes a region corresponding to positions chr6:32024461-chr6:32043719 and chr6:31991723-chr6:32010985 of reference genome hg38. In some embodiments, the RCCX region includes a region corresponding to positions chr6:31992238-chr6:32011496 and chr6:31959500-chr6:31978762 of reference genome hg19.

Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA). Sequence reads can be, for example, 50, 60, 70, 80, 90, 100, 110, 120, 130, 140, 150, 160, 170, 180, 190, 200, 400, 400, 500, 600, 700, 800, 900, 1000, 1250, 1500, 1750, 2000, or more base pairs (bps) in length each. For example, sequence reads are about 100 base pairs to about 1000 base pairs in length each. The sequence reads can comprise paired-end sequence reads. The sequence reads can comprise single-end sequence reads. The sequence reads can be generated by whole genome sequencing (WGS). The WGS can be clinical WGS (cWGS). The sample can comprise cells, cell-free DNA, cell-free fetal DNA, amniotic fluid, a blood sample, a biopsy sample, or a combination thereof.

In some embodiments, the sequence reads are obtained by aligning the reads to the RCCX region of a reference sequence. For example, sequence reads may be aligned to a reference genome as depicted in block 230 of FIG. 2A. In some embodiments, the sequence reads are obtained by aligning a first plurality of sequence reads generated from a sample to a reference genome sequence to obtain a second plurality of sequence reads which align to the RCCX region in the reference genome sequence. In some embodiments, a computing system stores the first plurality of sequence reads in memory. The computing system may load the first plurality of sequence reads into memory. A sequence read can be aligned to the RCCX region in the reference sequence with an alignment quality score of zero or more. A sequence read can be aligned to either copy of the RCCX module in the reference sequence with an alignment quality score of about zero (for example, when a sequence is aligned to a region where the gene and the gene paralog are highly homologous) or more.

In some embodiments, the sequence reads are obtained from a digital file containing sequencing information. In some embodiments, the digital file is on a computer storage medium (such as a computer hard drive, for example a spinning magnetic disk drive or a solid state drive). In some embodiments, the digital file is stored in the format of a BAM, FASTQ, SAM, CRAM, or VCF file.

### Estimating a Copy Number of the RCCX Region

In some embodiments, the methods and systems disclosed herein include a step of estimating a copy number of the RCCX region of the human genome in the nucleic acid sample from the aligned sequence reads, such as is depicted in block 240 of FIG. 2A.

In some embodiments, estimating a copy number of the RCCX region of the human genome comprises counting sequence reads which align to the RCCX region of the human genome. For example, sequence reads may have been previously aligned to a reference sequence as described. In some embodiments, estimating a copy number of the RCCX region of the human genome comprises counting sequence reads which align to a C4A gene, a CYP21A1P gene, a TNXA gene, a C4B gene, a CYP21A2 gene, and/or a TNXB gene in the human genome. In some embodiments, sequence reads are counted which align to either copy of the RCCX module (such as either copy of a gene within the RCCX region).

In some embodiments, estimating a copy number of the RCCX region of the human genome comprises counting sequence reads which align to a region corresponding to positions chr6:32024461-chr6:32043719 of reference genome hg38, chr6:31991723-chr6:32010985 of reference genome hg38, chr6:31992238-chr6:32011496 of reference genome hg19, and/or chr6:31959500-chr6:31978762 of reference genome hg19. In some embodiments, sequence reads are counted if they align to one or more sites within the aforementioned positions.

In some embodiments, estimating the copy number includes a step of normalizing the count of the sequence reads which align to the RCCX region of the human genome. In some embodiments, the sequence read count is normalized by the length of the RCCX region. In some embodiments, the read count may be normalized by the length of the region and against a set of 3000 genomic regions of 2000 bp expected to be consistently diploid across populations. In some embodiments, determining the normalized count of the sequence reads aligned to the RCCX regions comprises normalization using (1a) a depth of the sequence reads aligned to the RCCX region, (1b) a length of each of the RCCX region (such as a length of each RCCX module), (2a) a depth of sequence reads aligned to diploid regions, and (2b) a length of each of the diploid regions.

In some embodiments, estimating the copy number includes a step of GC-correcting the sequence read count. For example, in some embodiments, the sequence read count (for example, a sequence read count normalized by length of the region) for the RCCX region is pooled together with sequence read counts (for example, a sequence read count normalized by length of the region) for diploid regions including about 3,000 distinct 2 kb regions. Normalizing the count of sequence reads which align to the RCCX region by the count of sequence reads which align to diploid regions may, in some embodiments, correct for bias in sequencing coverage due to variable GC content among different regions. For example, the count of sequence reads aligned to each of the one or more target regions may be corrected for GC content using (1) a GC content of each of the RCCX region and (2) a GC content of each of diploid regions. In some embodiments, a normalized and/or GC-corrected copy number is determined for the RCCX region.

In some embodiments, estimating the copy number comprises binning the normalized count of the sequence reads which align to the RCCX region of the human genome using a Gaussian mixture model. For example, a Gaussian mixture model may be used to infer the most likely copy number of RCCX region based on the observed normalized depth signal.

The total copy number can be, for example, 1, 2, 3, 4, 5, 6, 7, 8, 9, 10, or more copies. The Gaussian mixture model can comprise a one-dimensional Gaussian mixture model. The plurality of Gaussians of the Gaussian mixture model can represent integer copy numbers, for example, 0 to 5, 0 to 6, 0 to 7, 0 to 8, 0 to 9, 0 to 10, 0 to 11, 0 to 12, 0 to 13, 0 to 14, or 0 to 15. For example, the plurality of Gaussians of the Gaussian mixture model can represent integer copy numbers from 0 to 10. A mean of each of the plurality of Gaussians can be the integer copy number represented by the Gaussian. A mean of each of the plurality of Gaussians can be the integer copy number represented by the Gaussian (such as copy numbers of 1, 2, 3, 4, 5, 6, 7, 8, 9, 10, 11, 12, 13, 14, 15, or more). The standard deviation of a Gaussian can be or be about, for example, 0.1, 0.2, 0.3, 0.4, 0.5, 0.6, 0.7, 0.8, 0.9, 1, or more. The plurality of Gaussians of the Gaussian mixture model can comprise, for example, 5, 6, 7, 8, 9, 10, 11, 12, 13, 14, 15, 16, or more, Gaussians. For example, the plurality of Gaussians of the Gaussian mixture model can comprise 5 Gaussians.

To estimate a copy number of the RCCX region, the computing system can determine the copy number using a Gaussian mixture model and a predetermined posterior probability threshold, given the normalized number of the sequence reads aligned to the RCCX region. The predetermined posterior probability threshold can be, for example, 0.7, 0.75, 0.8, 0.85, 0.95, or more. In some embodiments, the predetermined posterior probability threshold is 0.95.

### Pre-Determined Differentiating Sites

In some embodiments, the methods and systems disclosed herein use pre-determined differentiating sites. In some embodiments, the pre-determined differentiating sites include sites where the sequence of the CYP21A2 gene and the CYP21A1P pseudogene differ. In some embodiments, the sequence of the CYP21A2 gene and the CYP21A1P pseudogene differ at the pre-determined differentiating site in at least 90%, at least 95%, at least 97%, at least 98%, or at least 99% of a population of nucleic acid samples.

In some embodiments, the plurality of pre-determined differentiating sites comprise a site corresponding to a position selected from chr6:32038514, chr6:32038844, chr6:32039015, chr6:32039081, chr6:32039128, chr6:32039132, chr6:32039143, chr6:32039426, chr6:32039548, chr6:32039802, chr6:32039807, chr6:32039810, chr6:32039816, chr6:32040110, chr6:32040182, chr6:32040216, chr6:32040421, or chr6:32040535 of the CYP21A2 gene or a corresponding position in pseudogene CYP21A1P, in reference genome hg38.

In some embodiments, the plurality of pre-determined differentiating sites comprise a site corresponding to a position selected chr6:32006291, chr6:32006621, chr6:32006792, chr6:32006858, chr6:32006905, chr6:32006909, chr6:32006920, chr6:32007203, chr6:32007325, chr6:32007579, chr6:32007584, chr6:32007587, chr6:32007593, chr6:32007887, chr6:32007959, chr6:32007993, chr6:32008198, or chr6:32008312 of the CYP21A2 gene or a corresponding position in pseudogene CYP21A1P, in reference genome hg19.

The below table describes 18 pre-determined differentiating sites, 11 of which are pathogenic gene conversion variants. The positions listed in the table below are from chromosome 6 of reference genome hg38.

TABLE 1

| CYP21A2 | CYP21A2 | CYP21A1P | CYP21A1P | |
|---|---|---|---|---|
| Position | Base | Position | Base | Site/Variant |
| 32038514 | c | 32005780 | t | NM_000500.9: c.92C > T |
| 32038844 | a | 32006110 | c | g.32038844 |
| 32039015 | g | 32006287 | t | g.32039015 |
| 32039081 | c, a | 32006353 | g | NM_000500.9: c.293-13C > G |
| 32039128 | c | 32006400 | g | g.32039128 |
| 32039132 | GGAGACT | 32006404 | GTCTCTG | NM_000500.9: c.332_339del |
| 32039143 | c | 32006407 | t | g.32039143 |
| 32039426 | t | 32006690 | a | NM_000500.9: c.518T > A |
| 32039548 | c | 32006812 | g | g.32039548 |
| 32039802 | t | 32007066 | c | g.32039802 |
| 32039807 | t | 32007071 | a | NM_000500.9: c.710T > A |
| 32039810 | t | 32007074 | a | NM_000500.9: c.713T > A |
| 32039816 | t | 32007080 | a | NM_000500.9: c.719T > A |
| 32040110 | G | 32007374 | T | NM_000500.9: c.844G > T |
| 32040182 | GTTTTTTTGC | 32007446 | GTTTTTTTTG | NM_000500.9: c.923dupT |
| 32040216 | g | 32007481 | c | g.32040216 |
| 32040421 | c | 32007686 | t | NM_000500.9: c.955C > T |
| 32040535 | c | 32007800 | t | NM_000500.9: c.1069C > T |

In another aspect, disclosed herein are methods and systems for identifying a plurality of pre-determined differentiating sites. In some embodiments, the method comprises identifying single-base differences between the sequence of the CYP21A2 and CYP21A1P genes in a reference sequence. For example, a reference sequence of the CYP21A2 gene may be compared with a reference sequence of a CYP21A1P gene by aligning the sequences to each other and noting all sites with single base differences between the two gene sequences. The positions of those differentiating sites in both CYP21A2 and CYP21A1P genes may then be stored to an electronic storage. For example, a digital file may be created including a list of the single base differences.

In some embodiments, the method includes selecting, as differentiating sites, single-base differences which are fixed across a population. For example, the method may include, for a plurality of nucleic acid samples (such as a plurality of nucleic acid samples from a population of individuals), receiving a plurality of sequence reads which align to the CYP21A2 and CYP21A1P genes. In some embodiments, the plurality of nucleic acid samples are derived from individuals of a population, such as more than 100, more than 500, more than 1,000, more than 5,000, or more than 10,000 individuals. In some embodiments, the plurality of samples are taken from the 1000 Genomes Project. In some embodiments, the population is a diverse population, such as a genetically diverse population including individuals from a plurality of ethnic groups, such as to account for differences in population types and increase the likelihood that single-base differences do not comprise differences due to population type. The method may further include, for each of the plurality of nucleic acid samples, estimating a gene-specific copy number for the CYP21A2 gene and a copy number for the CYP21A1P gene. The method may further include selecting a subset of nucleic acid samples of the plurality of nucleic acid samples, wherein the subset of nucleic acid samples comprises nucleic acid samples which are estimated to be diploid for the CYP21A2 gene and diploid for the CYP21A1P gene (such as using only the data from samples which are estimated to not contain a recombination event between the CYP21A2 gene and the CYP21A1P gene). The method may further include selecting single-base differences which have copy numbers consistent with diploidy for the CYP21A2 gene and the CYP21A1P gene in at least 90%, at least 95%, at least 97%, at least 98%, or at least 99% of the nucleic acid samples of the subset of nucleic acid samples.

The method may further include creating a digital file which lists the positions of the selected single base differences, thereby generating a digital file including a plurality of pre-determined differentiating sites. In some embodiments, the digital file is on a computer storage medium (such as a computer hard drive, for example a spinning magnetic disk drive or a solid state drive). In some embodiments, the digital file is stored in the format of a BAM, SAM, FASTQ, CRAM, JSON or VCF file. The digital file may include information for the pre-determined differentiating sites such as the chromosome name where the pre-determined differentiating site is located, a 1-based inclusive start position in CYP21A1P, the expected base sequences for a CYP21A1P read mapped to the start position in CYP21A1P, a 1-based inclusive start position in CYP21A2, the expected base sequences for a CYP21A2 read mapped to the start position in CYP21A2, the region of CYP21A1P corresponding to the CYP21A2 start position, a unique name for the pre-determined differentiating site, and/or the orientation of the pre-determined differentiating site given by the orientation of the gene.

### Constructing One or More Candidate Haplotypes

In some embodiments, the methods and systems construct one or more candidate haplotypes, such as is depicted in process block 250 of FIG. 2A. In some embodiments, the methods and systems phase a plurality of sequence reads which align to a CYP21A2 gene or a CYP21A1P gene of the human genome. In some embodiments, the sequence reads include at least two pre-determined differentiating sites of the CYP21A2 gene and the CYP21A1P gene. In some embodiments, phasing the pre-determined differentiating sites includes constructing one or more candidate haplotypes based on all sequenced bases at a first pre-determined differentiating site, and extending the one or more candidate haplotypes to a second pre-determined differentiating site by aligning sequence reads of the CYP21A2 gene or CYP21A1P gene.

In some embodiments, constructing the one or more candidate haplotypes comprises identifying at least one seed sequence read from the plurality of sequence reads. In some embodiments, the seed sequence read aligns to a CYP21A2 gene or a CYP21A1P gene and includes at least two pre-determined differentiating sites of the CYP21A2 gene and the CYP21A1P gene. In some embodiments, the seed sequence read is selected from a 5′ seed sequence read, a center sequence read, and a 3′ seed sequence read. For example, in block 2520 of FIG. 2B, a 5′ seed sequence read, a center seed sequence read, or a 3′ seed sequence read is identified. In some embodiments, constructing the one or more candidate haplotypes comprises iteratively extending at least one seed sequence read in either a 5′ direction or a 3′ direction by aligning the sequence reads using the pre-determined differentiating sites. For example, in block 2530 of FIG. 2B, a seed sequence read is extended by alignment along pre-determined differentiating sites.

For example, candidate haplotypes may be formed from all sequenced bases at the first pre-determined differentiating site. For example, two candidate haplotypes may be formed if two bases are possible at a first pre-determined differentiating site based on basecalls from sequencing reads covering the first pre-determined differentiating site. In some embodiments, the haplotypes are then extended to the next pre-determined differentiating site by considering all sequencing reads that can be uniquely assigned to a single candidate haplotype. In some embodiments, if these sequencing reads support only a single base at the next differentiating site for a given candidate haplotype, then the haplotype is extended with that base. In some embodiments, when a candidate haplotype can be extended by two possible bases at the second pre-determined differentiating site, then both possible extended haplotypes are included in the set of candidate haplotypes, growing the set by 1. In some embodiments, subsequent extension steps are performed at a third pre-determined differentiating site, and the steps may be repeated until all sites have been processed. In some embodiments, this process yields a set of candidate haplotypes based on the bases observed at the plurality of pre-determined differentiating sites.

In some embodiments, the process can be run more than once using an alternate differentiating site as the starting point with extension performed in either the 3′ or 5′ direction along the haplotype. For example, in decision state 2540 of FIG. 2B, the system may determine whether extension steps should be performed for any additional seed sequence reads. In some embodiments, when the process is run multiple times with alternative starting differentiating sites and/or extension directions, a final run of the process may be performed to merge the partial candidate haplotypes formed from the previous runs of the process. For example, instead of using the original sequencing reads as input during this final run of the process, the partial candidate haplotypes from the previous runs of the process are used as if they were the input sequencing reads. For example, in block 2550 of FIG. 2B, partial candidate haplotypes are assembled into complete candidate haplotypes.

For example, the embodiment of FIG. 3 schematically depicts a 5′ seed sequence read 310, a center sequence read 320, and a 3′ seed sequence read 330. Each seed sequence read aligns to the CYP21A2 gene or the CYP21A1P gene and contains at least two pre-determined differentiating sites. In the depiction of FIG. 3, each site may include a “1” allele or a “2” allele. In the embodiment of FIG. 3, each seed sequence read is extended in a 3′ direction and/or 5′ direction using other sequence reads which align to the CYP21A2 gene or the CYP21A1P gene and contain at least two pre-determined differentiating sites. In the embodiment of FIG. 3, partial haplotypes 340 are constructed, which are then extended with other of the partial haplotypes 340 using the alleles at the pre-determined differentiating sites to generate a final candidate haplotype 350.

In some embodiments, a computing system constructs one or more candidate haplotypes originating from CYP21A2 gene or CYP21A1P gene, comprising a plurality of pre-determined differentiating sites using sequence reads aligned to the CYP21A2 gene or CYP21A1P gene, comprising the plurality of pre-determined differentiating sites. For example, a sequence read can be aligned to the reference sequence such that the sequence read overlaps a pre-determined differentiating site. A sequence read can be aligned to the region of CYP21A2 gene, or the corresponding region of the CYP21A1P gene, comprising the plurality of pre-determined differentiating sites with an alignment quality score of zero or more.

To phase the one or more haplotypes originating from the CYP21A2 gene or the CYP21A1P gene, the computing system can analyze linkage information between the pre-determined differentiating sites of the plurality of pre-determined differentiating sites using sequence reads aligned to the CYP21A2 or the CYP21A1P region, comprising the plurality of pre-determined differentiating sites. To phase the one or more haplotypes originating from CYP21A2 gene or CYP21A1P gene, the computing system can phase the one or more haplotypes originating from CYP21A2 gene or CYP21A1P gene using sequence reads aligned to two or more of the plurality of pre-determined differentiating sites.

In some embodiments, the one or more candidate haplotypes cover one or more breakpoints of the recombination event. For example, the one or more candidate haplotypes may cover one breakpoint, two breakpoints, three breakpoints, four breakpoints, five breakpoints, or more of a recombination event.

### Detecting a Recombination Event

In some embodiments, the methods and systems detect a recombination event between the CYP21A2 gene and the CYP21A1P gene based on the estimated copy number of the RCCX region of the human genome and based on the one or more candidate haplotypes. For example, the methods and systems may estimate a probability of a recombination event between the CYP21A2 gene and the CYP21A1P gene based on the estimated copy number of the RCCX region of the human genome and based on the one or more candidate haplotypes.

In some embodiments, a recombination event may be detected based on deviation from an estimated RCCX copy number of 4 and/or if at least one candidate haplotype contains both a CYP21A2-specific base and a CYP21A1P-specific base across the pre-determined differentiating sites. For example, in some embodiments, a deletion recombination event is detected if an estimated copy number of the RCCX region is less than or equal to three, and/or if a deletion recombinant variant is detected among the one or more candidate haplotypes.

For example, in the embodiment of FIG. 3, the candidate haplotype “2221111111121” may indicate a breakpoint between the first three pre-determined differentiating sites, which include the pseudogene CYP21A1P allele at those sites, and the fourth pre-determined differentiating sites, which begins a string of “1” s, indicating CYP21A2 gene alleles at those sites. Thus, a recombinant variant may be detected based on the candidate haplotype.

In some embodiments, a recombination event is not detected if the estimated RCCX copy number is 4 and if the one or more candidate haplotypes do not indicate a recombination event (for example, each candidate haplotype only includes all CYP21A2-specific bases or all CYP21A1P-specific bases across the pre-determined differentiating sites).

In some embodiments, the methods and systems include a step of making a variant call for the recombination event. In some embodiments, the methods and systems disclosed herein further include a step of creating a digital file including a variant call. In some embodiments, the file includes an estimated integer copy number for each of the one or more target regions, a float copy number for each of the one or more target regions, and a copy number genotype. In some embodiments, the digital file is on a computer storage medium (such as a computer hard drive, for example a spinning magnetic disk drive or a solid state drive). In some embodiments, the digital file is stored in the format of a BAM, FASTQ, SAM, CRAM, JSON, or VCF file. In some embodiments, the digital file is a VCF file or a JSON file.

In some embodiments, the digital file includes one or more candidate haplotypes. In some embodiments, the digital files includes an RCCX copy number. In some embodiments, the digital file includes information about whether a breakpoint was detected. In some embodiments, a breakpoint is detected based on the RCCX copy number and the one or more candidate haplotypes.

### Methods of Detecting Variants in a RCCX Region

In another aspect, disclosed herein are methods and systems of detecting one or more single-nucleotide variants or indels in a RCCX region in a nucleic acid sample. In some embodiments, the methods and systems determine sequence reads from the nucleic acid sample. For example, sequence reads may be determined as previously described herein with reference to methods and systems of detecting a recombination event between a CYP21A2 gene and a CYP21A1P gene.

In some embodiments, the methods and systems obtain sequence reads which align to a site of a single-nucleotide variant or indel in a CYP21A2 gene or a CYP21A1P gene of a human genome in the nucleic acid sample. For example, sequence reads may be aligned to a reference genome as previously described herein with reference to methods and systems of detecting a recombination event between a CYP21A2 gene and a CYP21A1P gene. Sequence reads may be collected which align to either the CYP21A2 gene or the CYP21A1P gene in the reference sequence, including sequence reads with low or zero mapping quality. In some embodiments, the sequence reads are derived from short-read sequencing systems or processes. In some embodiments, the short-read sequence reads are about 75 bp to about 500 bp in length. In other embodiments, the short-read sequence reads are 200 bp to about 400 bp in length.

In some embodiments, the methods and systems count sequence reads which contain a base corresponding to an alternative allele at the site of the single-nucleotide variant or indel. In some embodiments, counting sequence reads comprises counting both sequence reads which align to the CYP21A2 gene (and which include the site of the single-nucleotide variant or indel) and sequence reads which align to the CYP21A1P gene (and which include the site of the single-nucleotide variant or indel). In some embodiments, the sequence read count may be normalized and GC-corrected as previously described herein with reference to methods and systems of detecting a recombination event between a CYP21A2 gene and a CYP21A1P gene.

In some embodiments, the methods and systems create a digital file including a variant call corresponding to the single-nucleotide variant or indel (collectively, “small variant”). In some embodiments, the small variant will be reported if a significant portion of sequence reads support the alternative allele. For example, the small variant may be reported if about 10% or more, about 20% or more, about 30% or more, about 40% or more, about 50% or more, about 60% or more, about 70% or more, or about 80% or more, or about 90% or more sequence reads which cover the small variant contain a basecall corresponding to an alternative allele at the site of the small variant, as compared to a reference allele at the site. In some embodiments, the small variant may be reported if one or more, two or more, three or more, four or more, five or more, six or more, seven or more, eight or more, nine or more, or ten or more sequence reads contain an alternative allele at the site of the variant.

In some embodiments, sequence reads which include an alternative allele, and sequence reads which contain a reference allele, are counted. In some embodiments, an integer copy number is estimated for an alternative or variant allele based on a) a combined count of sequence reads covering corresponding positions of the small variant in CYP21A2 and CYP21A1P, b) a count of reads supporting reference alleles, and c) a count of reads supporting alternative alleles.

In some embodiments, the variant call is not specific to the CYP21A2 gene or the CYP21A1P gene. For example, in some embodiments, the variant call is not assigned to CYP21A2 or CYP21A1P or phased into one of the candidate haplotypes described further herein. In some embodiments, a small variant may be farther than one sequence read length (such as farther than 100 bp, 150 bp, 200 bp, 250 bp, 400 bp, 450 bp, or more) away from the one or more target regions described herein. In some embodiments, making a variant call ambiguous to CYP21A2 or CYP21A1P advantageously allows a user to detect one or more single-nucleotide variants or indels in a RCCX region in a nucleic acid sample while more efficiently using computing power and memory, as a detected small variant does not need to be phased into a candidate haplotype, and the methods and systems do not require that sequence reads are further analyzed to determine whether a small variant is assigned to CYP21A2 or CYP21A1P. In some embodiments, detecting a small variant in region-ambiguous manner improves computational resource efficiency and enables high precision and recall on discovering the variant allele, as compared to de-novo small variant calling or calling a small variant and phasing the small variant into a region or a baplotype, which require a much more complex process, are much less computationally efficient, and potentially provide less precision or recall for the variant of interest.

In some embodiments, a variant call that is ambiguous to the CYP21A2 or CYP21A1P genes advantageously allows a user to detect a small variant using short-read sequencing. Without being bound by theory, in some embodiments, short-read sequencing reads (such as sequence reads that include about 75-500 bp) over the CYP21A2 or CYP21A1P genes do not contain enough information to uniquely place the small variant and the user does not necessarily need to know the unique placement of the variant. In some embodiments, an advantage of making a region-ambiguous call is that the user avoids the need to perform more extensive sequencing assays, such as long-read sequencing assays. The information required can be obtained from the same whole genome sequencing (WGS) assay used to perform variant calling on the rest of the genome.

In some embodiments, once a variant call ambiguous to CYP21A2 or CYP21A1P has been made, the placement of the single-nucleotide variant or indel in the CYP21A2 gene or the CYP21A1P gene can be confirmed with orthogonal (long-read) sequencing methods known to those of skill in the art. For example, after a single-nucleotide variant or indel is detected in a manner not specific to the CYP21A2 gene or the CYP21A1P gene, additional sequencing such as orthogonal techniques are used to confirm the variant call and/or phase the variant into regions.

In some embodiments, the single-nucleotide variant or indel includes

| | |
|---|---|
| NM_000500.9: c.60G > A, | NM_000500.9: c.92C > A, |
| NM_000500.9: c.111del, | NM_000500.9: c.159_160del, |
| NM_000500.9: c.169G > A, | NM_000500.9: c.274A > G, |
| NM_000500.9: c.432_339del, | NM_000500.9: c.418G > A, |
| NM_000500.9: c.421G > A, | NM_000500.9: c.515T > A, |
| NM_000500.9: c.710_719delinsACGAGGAGAA, | NM_000500.9: c.850A > G, |
| NM_000500.9: c.874G > A, | NM_000500.9: c.922T > G, |
| NM_000500.9: c.923_924dup, | NM_000500.9: c.952C > T=, |
| NM_000500.9: c.955C > G, | NM_000500.9: c.1042G > A, |
| NM_000500.9: c.1051G > A, | NM_000500.9: c.1066C > T=, |
| NM_000500.9: c.1070G > A, | NM_000500.9: c.1096C > T, |
| NM_000500.9: c.1118G > A, | NM_000500.9: c.1136T > A, |
| NM_000500.9: c.1226G > A, | NM_000500.9: c.1273G > A, |
| NM_000500.9: c.1274G > T, | NM_000500.9: c.1279C > T, |
| NM_000500.9: c.1357C > T=, | NM_000500.9: c.1360C > T, |
| NM_000500.9: c.1444C > T, | NM_000500.9: c.1450dup, |
| or NM_000500.9: c.1451G > A. | |

The below table describes small variant sites, including 8 indels and 25 single-nucleotide variants. The positions listed in the table below are from chromosome 6 of reference genome hg38.

TABLE 2

| CYP21A2 | CYP21A2 | CYP21A1P | Variant | |
|---|---|---|---|---|
| position | base | position | allele | Variant ID |
| 32038482 | G | 32005748 | A | NM_000500.9:c.60G > A |
| 32038514 | C, T | 32005780 | A | NM_000500.9:c.92C > A |
| 32038532 | TCTTG | 32005798 | TTTG | NM_000500.9:c.111del |
| 32038579 | ACTCA | 32005845 | ACA | NM_000500.9:c.159_160del |
| 32038591 | G | 32005857 | A | NM_000500.9:c.169G > A |
| 32038793 | A | 32006059 | G | NM_000500.9:c.274A > G |
| 32039130 | TGGGAG | 32006402 | TGGAG | NM_000500.9:c.332_339del |
| 32039219 | G | 32006483 | A | NM_000500.9:c.418G > A |
| 32039222 | G | 32006486 | A | NM_000500.9:c.421G > A |
| 32039423 | T | 32006687 | A | NM_000500.9:0.515T > A |
| 32039802 | TCACAGCAG | 32007071 | TCACATCGTG | NM_000500.9:c.710_719del |
| | CTGAGG | | GAGAT | insACGAGGAGAA |
| 32040116 | A | 32007380 | G | NM_000500.9:c.850A > G |
| 32040140 | G | 32007404 | A | NM_000500.9:c.874G > A |
| 32040182 | GTTTTTTTGC | 32007446 | GTTTTTGTGC | NM_000500.9:c.922T > G |
| 32040182 | GTTTTTTTGC | 32007446 | GTTTTTTTGTG | NM_000500.9:c.923_924dup |
| | | | C | |
| 32040418 | C | 32007683 | T | NM_000500.9:c.952C > T= |
| 32040421 | C, T | 32007686 | G | NM_000500.9:c.955C > G |
| 32040508 | G | 32007773 | A | NM_000500.9:c.1042G > A |
| 32040517 | G | 32007782 | A | NM_000500.9:c.1051G > A |
| 32040532 | C | 32007797 | T | NM_000500.9:c.1066C > T= |
| 32040536 | G | 32007801 | C | NM_000500.9:c.1070G > A |
| 32040562 | C | 32007827 | T | NM_000500.9:c.1096C > T |
| 32040584 | G | 32007849 | A | NM_000500.9:c.1118G > A |
| 32040685 | T | 32007950 | A | NM_000500.9:c.1136T > A |
| 32040872 | G | 32008137 | A | NM_000500.9:c.1226G > A |
| 32040919 | G | 32008184 | A | NM_000500.9:c.1273G > A |
| 32040920 | G | 32008185 | T | NM_000500.9:c.1274G > T |
| 32040925 | C | 32008190 | T | NM_000500.9:c.1279C > T |
| 32041003 | C | 32008268 | T | NM_000500.9:c.1357C > T= |
| 32041006 | C | 32008271 | T | NM_000500.9:c.1360C > T |
| 32041090 | C | 32008355 | T | NM_000500.9:c.1444C > T |
| 32041092 | GCCCCGGGG | 32008357 | GCCCCCGGGG | NM_000500.9:c.1450dup |
| | GA, | | GA | |
| | GCCCCCGGG | | | |
| | A, | | | |
| | GCCCCCGGG | | | |
| | GA, | | | |
| | GCCCCAGGG | | | |
| | GA | | | |
| 32041092 | GCCCCGGGG | 32008357 | GCCCCAGGGG | NM_000500.9:c.1451G>A |
| | GA, | | A | |
| | GCCCCCGGG | | | |
| | GGA, | | | |
| | GCCCCCGGG | | | |
| | A, | | | |
| | GCCCCCGGG | | | |
| | GA | | | |

In some embodiments, the methods and systems disclosed herein further include a step of creating a digital file including a variant call. In some embodiments, the file includes, for each single-nucleotide variant or indel, a reference for the small variant, a count of sequence reads supporting an alternative allele, and a count of sequence reads supporting a reference allele. In some embodiments, the digital file is on a computer storage medium (such as a computer hard drive, for example a spinning magnetic disk drive or a solid state drive). In some embodiments, the digital file is stored in the format of a BAM, SAM, CRAM, FASTQ, JSON, or VCF file. In some embodiments, the digital file is a VCF file or a JSON file. In some embodiments, the digital file also includes recombinant event detection information as described herein.

### Embodiments of Sequencing Systems

FIG. 4A illustrates a diagram of an environment in which a recombination event detection system can operate in accordance with one or more implementations. The following paragraphs describe the recombination event detection system with respect to illustrative figures that portray example implementations and embodiments. For example, FIG. 4A illustrates a schematic diagram of a computing system 4000 in which a recombination event detection system 4106 operates in accordance with one or more implementations. As illustrated, the computing system 4000 includes one or more server device(s) 4102 connected to a user client device 4108, a local device 4118, and a sequencing device 4114 via a network 4112. The network 4112 can comprise any suitable network over which computing devices can communicate.

As shown in FIG. 4A, the computing system 4000 includes the server device(s) 4102. In various implementations, the server device(s) 4102 may generate, receive, analyze, store, and transmit digital data, such as data for nucleobase calls or sequenced nucleic-acid polymers. In some implementations, the server device(s) 4102 receive various data from the sequencing device 4114, such as data from a sample genome and/or sequence reads. The server device(s) 4102 may also communicate with the user client device 4108. In particular, the server device(s) 4102 can send data for sequence reads, direct nucleobase calls, nucleobase calls, and/or sequencing metrics to the user client device 4108.

As shown, the server device(s) 4102 includes a sequencing application 4110. In general, the sequencing application 4110 analyzes the data (such as call data) received from the sequencing device 4114 or elsewhere to determine nucleobase sequences for nucleic-acid polymers. For example, the sequencing application 4110 can receive raw data from the sequencing device 4114 and determine a nucleobase sequence for a sample genome or a nucleic-acid segment. In some implementations, the sequencing application 4110 determines the sequences of nucleobases in DNA and/or RNA segments or oligonucleotides.

As also shown, the sequencing application 4110 includes the recombination event detection system 4106. As described below, the recombination event detection system 4106 can detect a recombination event between a CYP21A2 gene and a CYP21A1P gene in a nucleic acid sample. For example, in some embodiments, the recombination event detection system 4106 receives sequence reads which align to a RCCX region of a human genome in the nucleic acid sample. The recombination event detection system 4106 further estimates a copy number of the RCCX region of the human genome in the nucleic acid sample from the aligned sequence reads. The recombination event detection system 4106 further constructs one or more candidate haplotypes by phasing a plurality of sequence reads which align to a CYP21A2 gene or a CYP21A1P gene of the human genome and which include at least two pre-determined differentiating sites of the CYP21A2 gene and the CYP21A1P gene. The recombination event detection system 4106 further detects a recombination event between the CYP21A2 gene and the CYP21A1P gene based on the estimated copy number of the RCCX region of the human genome and based on the one or more candidate haplotypes.

Moreover, while the recombination event detection system 4106 is described being implemented on the server device(s) 4102, as part of the sequencing application 4110, in some implementations, the recombination event detection system 4106 is implemented by (such as located entirely or in part) on the user client device 4108, the sequencing device 4114, and/or the local device 4118. As mentioned, in some implementations, the recombination event detection system 4106 is implemented by one or more other components of the computing system 4000, such as the sequencing device 4114. In particular, the recombination event detection system 4106 can be implemented in a variety of different ways across the server device(s) 4102, the network 4112, the user client device 4108, the local device 4118, and the sequencing device 4114.

As further shown in FIG. 4A, the computing system 4000 includes the user client device 4108. In various implementations, the user client device 4108 can generate, store, receive, and send digital data. In particular, the user client device 4108 can receive the data from the sequencing device 4114. As further illustrated, the user client device 4108 includes a sequencing application 4110. The sequencing application 4110 may be a web application or a native application stored and executed on the user client device 4108 (e.g., a mobile application, desktop application, or web application). The sequencing application 4110 can receive data from the sequencing application 4110 and/or recombination event detection system 4106. For example, the user client device 4108 can receive variant call files and/or alignment files from the sequencing application 4110.

The sequencing application 4110 can also include instructions that (when executed) cause the user client device 4108 to receive data from the recombination event detection system 4106 and present data from the sequencing device 4114 and/or the server device(s) 4102. Furthermore, the sequencing application 4110 can instruct the user client device 4108 to display data for variant calls, such as nucleobase calls and/or one or more candidate haplotypes. Indeed, the user client device 4108 can display nucleobase call results for a genome sample and/or an indication of a detected recombination event between a CYP21A2 gene and a CYP21A1P gene.

As further shown in FIG. 4A, the computing system 4000 includes the sequencing device 4114. In various implementations, the sequencing device 4114 can sequence a genomic sample or other nucleic-acid polymer. For example, the sequencing device 4114 analyzes nucleic-acid segments or oligonucleotides extracted from genomic samples to generate data either directly or indirectly on the sequencing device 4114. More particularly, the sequencing device 4114 receives and analyzes, within nucleotide-sample slides (such as flow cells), nucleic-acid sequences extracted from genomic samples. In one or more implementations, the sequencing device 4114 utilizes SBS to sequence a genomic sample or other nucleic-acid polymers. In addition to, or in the alternative to communicating across the network 4112, in some implementations, the sequencing device 4114 bypasses the network 4112 and communicates directly with the user client device 4108.

As further depicted in FIG. 4A, in some implementations, the server device(s) 4102 includes a distributed collection of servers, where the server device(s) 4102 include several server devices distributed across the network 4112 and located in the same or different physical locations. For instance, the server device(s) 4102 can be implemented, in whole or in part, on the local device 4118. To illustrate, the local device 4118 may implement the sequencing application 4110 and/or the recombination event detection system 4106. Further, the server device(s) 4102 and/or the local device 4118 can include a content server, an application server, a communication server, a web-hosting server, or another type of server.

The user client device 4108 illustrated in FIG. 4A can include various types of client devices. For example, in some implementations, the user client device 4108 includes non-mobile devices, such as desktop computers or servers, or other types of client devices. In various implementations, the user client device 4108 includes mobile devices, such as laptops, tablets, mobile telephones, or smartphones.

Though FIG. 4A illustrates the components of the computing system 4000 communicating via the network 4112, in certain implementations, the components of computing system 4000 can also communicate directly with each other, bypassing the network 4112. For instance, in some implementations, the user client device 4108 communicates directly with the sequencing device 4114. Additionally, in some implementations, the user client device 4108 communicates directly with the recombination event detection system 4106 and/or the server device(s) 4102. In some implementations, the user client device 4108 communicates directly with the local device 4118. Moreover, the recombination event detection system 4106 can access one or more databases housed on or accessed by the server device(s) 4102 or elsewhere in the computing system 4000.

FIG. 4B is a block diagram of an exemplary server device 4102 that may be used in connection with the illustrative computing system 4000 of FIG. 4A. The server device 4102 may be configured to detect a recombination event between a CYP21A2 gene and a CYP21A1P gene in a nucleic acid sample. The general architecture of the server device 4102 depicted in FIG. 4B includes an arrangement of computer hardware and software components. The server device 4102 may include many more (or fewer) elements than those shown in FIG. 4B. It is not necessary, however, that all of these generally conventional elements be shown in order to provide an enabling disclosure. As illustrated, the server device 4102 includes a processing unit 410, a network interface 420, a computer readable medium drive 430, an input/output device interface 440, a display 450, and an input device 460, all of which may communicate with one another by way of a communication bus. The network interface 420 may provide connectivity to one or more networks or computing systems. The processing unit 410 may thus receive information and instructions from other computing systems or services via a network. The processing unit 410 may also communicate to and from memory 470 and further provide output information for an optional display 450 via the input/output device interface 440. The input/output device interface 440 may also accept input from the optional input device 460, such as a keyboard, mouse, digital pen, microphone, touch screen, gesture recognition system, voice recognition system, gamepad, accelerometer, gyroscope, or other input device.

The memory 470 may contain computer program instructions (grouped as modules or components in some embodiments) that the processing unit 410 executes in order to implement one or more embodiments. The memory 470 generally includes RAM, ROM and/or other persistent, auxiliary or non-transitory computer readable media. The memory 470 may store an operating system 472 that provides computer program instructions for use by the processing unit 410 in the general administration and operation of the server device 4102. The memory 470 may store a reference genome 473, such as for use by the sequencing application 4110. The memory 470 may further include computer program instructions and other information for implementing aspects of the present disclosure.

For example, in one embodiment, the memory 470 includes a sequencing application 4110, which may include a recombination event detection system 4106. The recombination event detection system 4106 can perform the methods disclosed herein. In addition, memory 470 may include or communicate with the data store 490 and/or one or more other data stores that store one or more inputs, one or more outputs, and/or one or more results (including intermediate results) of detecting a recombination event between a CYP21A2 gene and a CYP21A1P gene in a nucleic acid sample of the present disclosure, such as the sequencing reads, the estimated copy number(s), one or more candidate haplotypes, and the variant call (for example, the detection of a recombination event) determined.

In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD-ROM, DVD-ROM, memory stick, flash drive, hard drive, SSD hard drive, server, mainframe storage system and the like.

In some embodiments, the methods may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (such as iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1

In the following example, recombination events between the CYP21A2 gene and the CYP21A1P gene were detected in a number of nucleic acid samples. The number of copies of the total RCCX region was identified and any recombinant CYP21A1P-CYP21A2 gene fusions were reported. Additionally, 33 small variants (single-nucleotide variants or indels) were detected in either the gene or the pseudogene. These variant calls included all CYP21A2 variants with multiple submitters that have been annotated in ClinVar17 as pathogenic or likely pathogenic.

### Total RCCX Copy Number

The number of copies of the total RCCX region was counted by counting reads belonging to both copies of the segmental duplication. In most cases, reads could not be unambiguously mapped to either copy of the repeat due to high sequence homology, but all reads placed in either copy were counted to provide a more accurate measure of the summed copy number of both regions. The region used for copy number calling covered most, but not all, of the RCCX region. The region used for copy number calling began after a polymorphic 6.4 kb HERV-K retrotransposon in introns of both C4A and C4B and extended 20 kb downstream to a 120 bp deletion in TNXA, including the entirety of the CYP21A2 gene. The region corresponded to positions chr6:32024461-chr6:32043719 and chr6:31991723-chr6:32010985 of reference genome hg38. The copy number calling subregion of RCCX was therefore large enough to reach any nonallelic homologous recombination events (which, affecting a whole copy of RCCX, are 30 kb in length). Read coverage was corrected for GC content by normalization against a panel of 3000 preselected 2 kb genomic sites with highly consistent diploid copy number. The normalized sequence read count was then binned using a Gaussian mixture model. This estimated copy number was an accurate estimate of the total copy number of the RCCX segmental duplication.

### Recombinant Variant Detection

A panel of 18 pre-determined differentiating sites across CYP21A2 were used to detect gene fusions between CYP21A1P and active CYP21A2. The sequence of the gene and the pseudogene differed at these pre-determined differentiating sites. The 18 pre-determined differentiating sites included chr6:32038514, chr6:32038844, chr6:32039015, chr6:32039081, chr6:32039128, chr6:32039132, chr6:32039143, chr6:32039426, chr6:32039548, chr6:32039802, chr6:32039807, chr6:32039810, chr6:32039816, chr6:32040110, chr6:32040182, chr6:32040216, chr6:32040421, and chr6:32040535 of the CYP21A2 gene or a corresponding position in pseudogene CYP21A1P, in reference genome hg38.

To identifying recombinant variants, haplotypes that occur within the genome were detected. Reads were collected that span the set of 18 pre-determined differentiating sites. Reads that spanned multiple (i.e., two or more) pre-determined differentiating sites were used to build connected haplotypes across the entire region. Reads containing pre-determined differentiating sites were collected and assembled into partial haplotypes from the 5′ end, center, and 3′ end of the gene. Partial haplotypes were then assembled into final complete haplotypes that spanned the full gene region. Transitions within the resulting haplotypes, from gene-allele to pseudogene-allele sequences, indicated either full chimeric gene fusions or smaller gene conversion events.

### Targeted Small Variant Detection

Other deleterious variations were detected for a set of 33 known sites, where the gene and pseudogene sequences were identical. Reads aligning to the gene or pseudogene for each of these sites were collected. The number of reads containing the reference allele, and any supporting the deleterious alternate allele, were counted and reported. The reads were then used to provide evidence for the presence or absence of the pathogenic allele in either the gene or the pseudogene. The 33 sites used were NM_000500.9:c.60G>A, NM_000500.9:c.92C>A, NM_000500.9:c.111del, NM 000500.9:c.159 160del, NM_000500.9:c.169G>A, NM_000500.9:c.418G>A, NM_000500.9:c.274A>G, NM_000500.9:c.332_339del, NM_000500.9:c.421G>A, NM_000500.9:c.515T>A, NM_000500.9:c.710 719delinsACGAGGAGAA, NM_000500.9:c.850A>G, NM_000500.9:c.874G>A, NM_000500.9:c.922T>G, NM_000500.9:c.923_924dup, NM_000500.9:c.1042G>A, NM_000500.9:c.1070G>A, NM_000500.9:c.952C>T=, NM_000500.9:c.955C>G, NM_000500.9:c.1051G>A, NM_000500.9:c.1066C>T=, NM_000500.9:c.1096C>T, NM_000500.9:c.1118G>A, NM_000500.9:c.1136T>A, NM_000500.9:c.1226G>A, NM_000500.9:c.1273G>A, NM_000500.9:c.1274G>T, NM_000500.9:c.1279C>T, NM_000500.9:c.1357C>T=, NM_000500.9:c.1360C>T, NM_000500.9:c.1444C>T, NM_000500.9:c.1450dup, and NM_000500.9:c.1451G>A.

Results: CAH Cases from the Radboud UMC (N=16, Cases)

WGS data was obtained for 16 congenital adrenal hyperplasia (CAH) cases with validation from Sanger sequencing or multiplex ligation-dependent probe amplification (MLPA). For each sample, recombination events between a CYP21A2 gene and a CYP21A1P gene were determined, and small variants were determined, using the targeted methods described above and were validated with the Sanger sequencing or MLPA. In each of these case genomes, the targeted methods accurately detected the total RCCX copy number and the pathogenic variants, including small variants, full gene deletions, and inactivating gene conversions.

The results of the validation for each of the 16 samples are summarized in the table below. In the table below, in each genome, the causal alleles and total RCCX copy number were reported. The targeted methods matched the MLPA/Sanger results in each case. All variant IDs are respective to the NM_000500.9 transcript.

TABLE 3

| | | Call | | Call | RCCX |
|---|---|---|---|---|---|
| ID | Allele 1 | match | Allele 2 | match | CN |
| 1 | Full deletion | ✓ | Full deletion | ✓ | 2 |
| 2 | Fusion: | ✓ | Fusion: | ✓ | 2 |
| | BEGIN - c.92C > T | | BEGIN - c.92C > T | | |
| 3 | c.293-13C > G | ✓ | Fusion: | ✓ | 2 |
| | | | c.92C > T - c.332_339del | | |
| 4 | c.293-13C > G | ✓ | Full deletion | ✓ | 3 |
| 5 | c.293-13C > G | ✓ | Fusion: | ✓ | 3 |
| | | | BEGIN - c.92C > T | | |
| 6 | c.518T > A | ✓ | c.518T > A | ✓ | 4 |
| 7 | c.518T > A | ✓ | c.518T > A | ✓ | 4 |
| 8 | c.1069C > T | ✓ | c.518T > A | ✓ | 4 |
| 9 | c.1360C > T | ✓ | c.1360C > T | ✓ | 4 |
| 10 | c.1069C > T | ✓ | c.421G > A | ✓ | 5 |
| 11 | c.844G > T | ✓ | c.518T > A | ✓ | 5 |
| 12 | c.844G > T | ✓ | Full deletion | ✓ | 5 |
| 13 | c.844G > T | ✓ | c.1360C > T | ✓ | 5 |
| 14 | c.293-13C > G | ✓ | Full deletion | ✓ | 6 |
| 15 | c.293-13C > G | ✓ | Full deletion | ✓ | 4 |
| 16 | Fusion: | ✓ | Fusion: | ✓ | 6 |
| | c.92C > T - c.332_339del | | c.92C > T - c.332_339del | | |

### Example 2

In the following example, recombination events between the CYP21A2 gene and the CYP21A1P gene were detected, along with small variants, in four nucleic acid samples using the methods described in Example 1.

The methods described in Example 1 were further validated with four sequenced cell lines, with MLPA or long-range PCR confirmation of CYP21A2 variants. These included a trio in which the proband, NA14734, was affected by the severe salt-wasting form of CAH. This was caused by full deletion of two copies of the RCCX segmental duplication and a complete loss of CYP21A2, as evidenced by MLPA validation. MLPA also revealed that both parents were carriers of CYP21A2 deletions, clarifying the inheritance of the deleterious genotype in the proband.

Using the methods described in Example 1, each of these genotypes were identified in the trio. The haplotypes generated by deletions of the RCCX module were constructed and the total RCCX copy number in each family member was estimated. The detailed information obtained from the candidate haplotypes also provided insight into the inherited alleles of the CYP21A1P pseudogene.

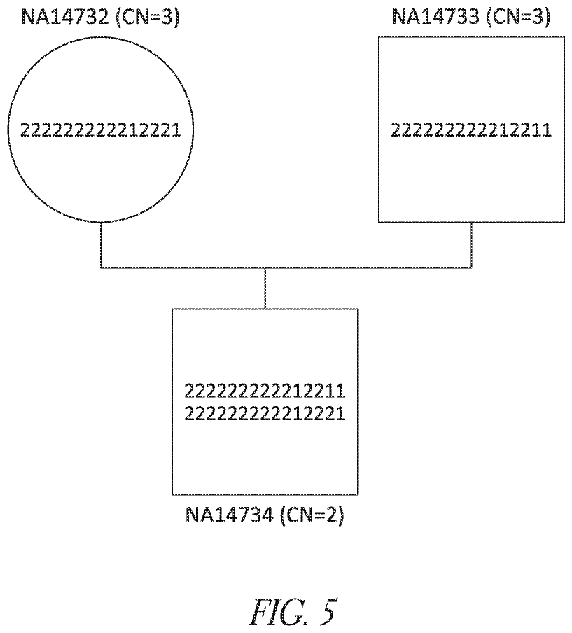

FIG. 5 schematically illustrates recombinant haplotypes constructed in a CAH case trio. In FIG. 5, each haplotype is simplified to a series of 1 or 2 identifiers, indicating the gene (1) or pseudogene (2) allele at each differentiating site. The haplotype of the CAH-affected proband NA14734 contained copies of the RCCX segmental duplication with the inactive pseudogene CYP21A1P allele at most sites, and no copies of the wildtype CYP21A2 gene. The most likely parental origins of the two RCCX copies in the proband were identified. Copy number calls of 3 in each parent also indicate risk of wildtype gene deletions. Each parent was identified as a possible CAH carrier due to decreased RCCX copy number. The proband, lacking any copies of the active gene, was identified as a likely CAH case.

The fourth CAH cell line (NA12217) was also a CAH case, although affected by the more moderate simple virilizing form of the disorder. In this genome, MLPA and long-range PCR validation identified a single deletion of one copy of RCCX and an exonic single-nucleotide variant, NM_000500.9:c.518T>A, with known CAH risk. Using the methods described in Example 1, an RCCX copy number was estimated and candidate haplotypes were constructed. The results are shown in the table below.

TABLE 4

| Sample | Call | RCCX CN | Haplotypes | Coriell Validation |
|---|---|---|---|---|
| Proband: | I173N + | 4 | 121111121111111111 | One haplotype has 30 kb |
| NA12217 | HET DEL | | 222222211111111111 | deletion |
| | | | 222222222222222221 | One haplotype has |
| | | | 222222222222222222 | I173N |

As seen in the above table, the NM_000500.9:c.518T>A variant was identified in one haplotype, the total RCCX copy number of four was estimated, and also a chimeric pseudogene-gene fusion likely derived from a recombination-mediated deletion was identified. This deletion event, in tandem with the total RCCX copy number of four, indicated that this genome represents the outcome of both deletion and duplication in the RCCX module.

The “121111121111111111” haplotype would be challenging to assemble with conventional methods but was unambiguous using the methods described in Example 1. The chimeric fusion haplotype structure was represented as “222222211111111111”, where “1” indicates the target gene allele and “2” indicates the pseudogene allele. The haplotype showed a clear delineation between consistent pseudogene alleles at the first seven differentiating sites, then conversion to consistent gene alleles at the final eleven sites, a refined representation of the fusion gene structure and deletion breakpoints.

### Example 3

In the following example, the total RCCX copy number was estimated for a number of nucleic acid samples (N=204) using the methods described in Example 1. The estimated RCCX copy number was compared to orthogonal sequencing results.

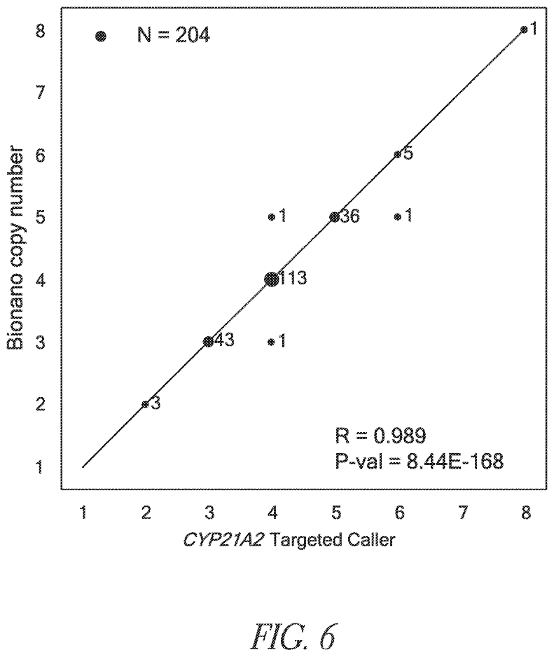

The RCCX total copy number calling results from the methods described in Example 1 was compared to RCCX copy number calls from the orthogonal Bionano Genomics optical mapping technology in 204 genomes from the 1000 Genomes Project cohort. The results are depicted in FIG. 6. Pearson's correlation coefficient and P-value are annotated at bottom-right of FIG. 6.

While optical mapping lacks the resolution to identify gene fusions or small variation, these call comparisons indicated the overall copy number calling accuracy of the methods described in Example 1 (“CYP21A2 Targeted Caller”). In 201 of 204 genomes, the copy number calls agreed, while in three genomes there was a disagreement of one RCCX copy. This concordance demonstrates the high accuracy of the methods described in Example 1 in recovering the correct copy number of the RCCX region.

### Example 4

In the following example, 33 small variants (single-nucleotide variants or indels) were tested in either the CYP21A2 gene or the CYP21A1P pseudogene using the methods described in Example 1. 3195 samples from the 1000 Genomes Project cohort were tested for the 33 small variants and results were reviewed. Eleven out of 3195 (0.3%) contained strong evidence for a targeted variant (at least two supporting sequence reads, from either the gene or pseudogene). While these variant calls are highly confident, they were not uniquely assigned to the gene or pseudogene, and were ambiguously assigned to either the gene or the pseudogene.

### Other Considerations

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, such as a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (such as X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or +1%. The term “substantially” is used to indicate that a result (such as a measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

The scope of the present disclosure is not intended to be limited by the specific disclosures of examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A computer-implemented method of detecting a recombination event between a CYP21A2 gene and a CYP21A1P gene in a nucleic acid sample, the method comprising:
receiving sequence reads which align to a RCCX region of a human genome in the nucleic acid sample;
estimating a copy number of the RCCX region of the human genome in the nucleic acid sample from the aligned sequence reads;
constructing one or more candidate haplotypes by phasing a plurality of sequence reads which align to a CYP21A2 gene or a CYP21A1P gene of the human genome and which include at least two pre-determined differentiating sites of the CYP21A2 gene and the CYP21A1P gene; and
detecting a recombination event between the CYP21A2 gene and the CYP21A1P gene based on the estimated copy number of the RCCX region of the human genome and based on the one or more candidate haplotypes.

2. The method of claim 1, wherein the one or more candidate haplotypes cover one or more breakpoints of the recombination event.

3. The method of claim 1, wherein constructing the one or more candidate haplotypes comprises identifying at least one seed sequence read from the plurality of sequence reads.

4. The method of claim 3, wherein the seed sequence read is selected from a 5′ seed sequence read, a center sequence read, and a 3′ seed sequence read.

5. The method of claim 3, wherein constructing the one or more candidate haplotypes comprises iteratively extending at least one seed sequence read in either a 5′ direction or a 3′ direction by aligning the sequence reads using the pre-determined differentiating sites.

6. The method of claim 1, wherein estimating a copy number of the RCCX region of the human genome comprises counting sequence reads which align to the RCCX region of the human genome.

7. The method of claim 6, wherein estimating a copy number of the RCCX region of the human genome comprises counting sequence reads which align to a C4A gene, a CYP21A1P gene, a TNXA gene, a C4B gene, a CYP21A2 gene, or a TNXB gene in the human genome.

8. The method of claim 7, wherein estimating a copy number of the RCCX region of the human genome comprises counting sequence reads which align to a region corresponding to positions chr6:32024461-chr6:32043719 of reference genome hg38, chr6:31991723-chr6:32010985 of reference genome hg38, chr6:31992238-chr6:32011496 of reference genome hg19, or chr6:31959500-chr6:31978762 of reference genome hg19.

9. The method of claim 6, wherein estimating the copy number comprises normalizing the count of the sequence reads which align to the RCCX region of the human genome.

10. The method of claim 9, wherein estimating the copy number comprises binning the normalized count of the sequence reads which align to the RCCX region of the human genome using a Gaussian mixture model.

11. The method of claim 1, further comprising making a variant call at a pre-determined differentiating site of the plurality of pre-determined differentiating sites.

12. The method of claim 1, further comprising making a variant call for the recombination event.

13. The method of claim 1, further comprising creating a digital file including a variant call.

14. The method of claim 1, further comprising creating a digital file including one or more candidate haplotypes.

15. The method of claim 1, wherein the plurality of pre-determined differentiating sites comprise a site corresponding to a position selected from chr6:32038514, chr6:32038844, chr6:32039015, chr6:32039081, chr6:32039128, chr6:32039132, chr6:32039143, chr6:32039426, chr6:32039548, chr6:32039802, chr6:32039807, chr6:32039810, chr6:32039816, chr6:32040110, chr6:32040182, chr6:32040216, chr6:32040421, or chr6:32040535 of the CYP21A2 gene or a corresponding position in pseudogene CYP21A1P, in reference genome hg38.

16. The method of claim 1, wherein the plurality of pre-determined differentiating sites comprise a site corresponding to a position selected chr6:32006291, chr6:32006621, chr6:32006792, chr6:32006858, chr6:32006905, chr6:32006909, chr6:32006920, chr6:32007203, chr6:32007325, chr6:32007579, chr6:32007584, chr6:32007587, chr6:32007593, chr6:32007887, chr6:32007959, chr6:32007993, chr6:32008198, or chr6:32008312 of the CYP21A2 gene or a corresponding position in pseudogene CYP21A1P, in reference genome hg19.

17. A computer-implemented method of detecting one or more single-nucleotide variants or indels in a RCCX region in a nucleic acid sample, the method comprising:
determining sequence reads from the nucleic acid sample;
obtaining sequence reads which align to a site of a single-nucleotide variant or indel in a CYP21A2 gene or a CYP21A1P gene of a human genome in the nucleic acid sample;
counting sequence reads which contain a base corresponding to an alternative allele at the site of the single-nucleotide variant or indel, wherein counting sequence reads comprises counting sequence reads which align to the CYP21A2 gene and sequence reads which align to the CYP21A1P gene; and
creating a digital file including a variant call corresponding to the single-nucleotide variant or indel, wherein the variant call is not specific to the CYP21A2 gene or the CYP21A1P gene.

18. The method of claim 17, wherein the one or more single-nucleotide variants or indels comprise NM_000500.9:c.60G>A, NM_000500.9:c.92C>A, NM_000500.9:c.111del, NM_000500.9:c.159_160del, NM_000500.9:c.169G>A, NM_000500.9:c.274A>G, NM_000500.9:c.421G>A, NM_000500.9:c.332_339del, NM_000500.9:c.418G>A, NM_000500.9:c.515T>A, NM_000500.9:c.710_719delinsACGAGGAGAA, NM_000500.9:c.874G>A, NM_000500.9:c.922T>G, NM_000500.9:c.955C>G, NM_000500.9:c.952C>T=, NM_000500.9:c.1066C>T=, NM_000500.9:c.1051G>A, NM_000500.9:c.1118G>A, NM_000500.9:c.1096C>T, NM_000500.9:c.1226G>A, NM_000500.9:c.1273G>A, NM_000500.9:c.850A>G, NM_000500.9:c.923_924dup, NM_000500.9:c.1042G>A, NM_000500.9:c.1070G>A, NM_000500.9:c.1136T>A, NM_000500.9:c.1274G>T, NM_000500.9:c.1279C>T, NM_000500.9:c.1357C>T=, NM_000500.9:c.1360C>T, NM_000500.9:c.1444C>T, NM_000500.9:c.1450dup, or NM_000500.9:c.1451G>A.

19. An electronic system for detecting a recombination event between a CYP21A2 gene and a CYP21A1P gene in a nucleic acid sample, comprising a processor configured to perform a method comprising:
receiving sequence reads which align to a RCCX region of a human genome in the nucleic acid sample;
estimating a copy number of the RCCX region of the human genome in the nucleic acid sample from the aligned sequence reads;
constructing one or more candidate haplotypes by phasing a plurality of sequence reads which align to a CYP21A2 gene or a CYP21A1P gene of the human genome and which include at least two pre-determined differentiating sites of the CYP21A2 gene and the CYP21A1P gene; and
detecting a recombination event between the CYP21A2 gene and the CYP21A1P gene based on the estimated copy number of the RCCX region of the human genome and based on the one or more candidate haplotypes.

20. (canceled)

21. The electronic system of claim 19, wherein the one or more candidate haplotypes cover one or more breakpoints of the recombination event.

22. The electronic system of claim 19, wherein constructing the one or more candidate haplotypes comprises identifying at least one seed sequence read from the plurality of sequence reads.

23. The electronic system of claim 22, wherein the seed sequence read is selected from a 5′ seed sequence read, a center sequence read, and a 3′ seed sequence read.

24. The electronic system of claim 22, wherein constructing the one or more candidate haplotypes comprises iteratively extending at least one seed sequence read in either a 5′ direction or a 3′ direction by aligning the sequence reads using the pre-determined differentiating sites.

25. The electronic system of claim 19, wherein estimating a copy number of the RCCX region of the human genome comprises counting sequence reads which align to the RCCX region of the human genome.
